(19) United States
(12) Patent Application Publication
(10) Pub. No.: US 2012/0041465 A1
(43) Pub. Date: Feb. 16, 2012

(54) DEVICES AND METHODS FOR TREATING GASTROINTESTINAL AND METABOLIC DISORDERS

(76) Inventor: Tidhar SHALON, Palo Alto, CA (US)

(21) Appl. No.: 13/280,418

(22) Filed: Oct. 25, 2011

Related U.S. Application Data
(63) Continuation of application No. PCT/IL2010/000347, filed on Apr. 29, 2010.

(51) Int. Cl. A61F 2/04

(52) U.S. Cl. 606/191

(57) ABSTRACT

A device for treating gastrointestinal and metabolic disorders is described. The device includes a device body which is present in the GI tract tissue of a subject and functions in treating gastrointestinal and metabolic disorders such as obesity, diabetes and reflux disease.

### RELATED APPLICATIONS

This application is a continuation of PCT Patent Application No. PCT/IL2010/000347 filed Apr. 29, 2010, which claims the benefit of priority under 35 USC 119(e) of U.S. Provisional Patent Application No. 61/174,019 filed Apr. 30, 2009, the contents of which are incorporated herein by reference in their entirety.

### FIELD AND BACKGROUND OF THE INVENTION

The present invention relates to devices and methods which can be used to treat gastrointestinal and metabolic disorders, for example obesity.

During the past 20 years, obesity among adults has risen significantly in the United States. The latest data from the National Center for Health Statistics show that 30 percent of U.S. adults 20 years of age and older—over 60 million people—are obese. Obesity requires long-term management; the goal of treatment is weight loss to improve, prevent occurrence of, or eliminate related health problems.

It is also well known that type 2 diabetes, which in itself is a growing epidemic, is directly related to obesity and that successful weight loss following bariatric surgery such as the Roux-en-Y procedure, successfully restores glycemic control to those patients, and markedly reduces their underlying diabetes. Therefore, successful treatment of obesity can also lead to improvements in metabolic disorders such as diabetes.

Numerous approaches for the treatment of obesity are known in the art, including drug treatment, surgical procedures and implantable devices.

Drugs for treatment of obesity fall into three general categories, appetite altering drugs such as dexfenfluramine or sibutramine which suppresses appetite by altering neurotransmitter release or uptake in the brain; metabolism-changing drugs such as Orlistat which prevents the action of lipases (enzymes that break down fat) produced in the pancreas; and drugs that increase energy output (‘thermogenic’ drugs) such as ephedrine and caffeine which stimulate weight loss by reducing appetite and perhaps by stimulating the body to produce more heat.

Although these drugs offer useful therapeutic effects, there remains a need for more effective obesity treatment drugs. Such a need will fuel tremendous commercial opportunity and so in the future drugs which target gastrointestinal or brain receptors for satiety, or block/mimic the action of satiety altering hormones and substances (such as ghrelin, CCK, PYY, obestatin, leptin, glucagons, neuropeptide Y and the like) might make their way to the market.

Two forms of surgery have been recommended by government consensus panels that can be performed to treat severe obesity. Both are for people with severe cases of obesity, over 100 lbs above ideal body weight (e.g. BMI>40 kg/m²), who have not had effective weight loss with diet, exercise or drugs.

Gastroplasty involves surgically reducing the size of the stomach, thus limiting food intake. Vertical band gastroplasty (VBG) is successful in more than 85% of patients, and weight loss is maintained over prolonged time periods (Barclay Obes Surg. 2004 November-December; 14(10):1415-8). Gastric bypass surgery (e.g. Roux en Y) creates a small stomach pouch and connects this pouch to the second portion of the intestines. Gastric bypass surgery can initially result in substantial weight loss, and approximately 80 percent of patients remain at least 10 percent below their preoperative body weight for 10 years after surgery. The efficacy of the procedure is probably due to the increased sense of fullness with a reduced gastric volume and the symptoms of “dumping” associated with the passage of gastric contents into the intestines, which act as deterrents to eating (Rosenbaum et al. Obesity NEJM Volume 337:396-407 Aug. 7, 1997 Number 6). Although gastric bypass surgery is highly effective, it carries a risk of morbidly and it is more extensive and difficult to perform than gastroplasty.

Numerous devices for altering satiety are also known in the art. Some devices restrict stomach size or food intake via bands [e.g. lap band et al. MJA 2005; 183 (6): 310-314] or space occupying elements [e.g. intra-stomach balloons—Obes Surg. 2005 September; 15(8):1161-4]. Others alter stomach or pyloric muscle activity via neuronal or muscular implanted electrodes (Shikora, Journal of gastrointestinal surgery Volume 8, Issue 4, Pages 408-412; Xu et al. Gastroenterology 2005; 128:43-50).

Although numerous treatment approaches are available at present, the most effective approach with the best long term effects is restricted to the treatment of severely obese people and in addition it requires complicated surgery which can lead to severe complications or death.

There is thus a widely recognized need for, and it would be highly advantageous to have, an eating behavior altering device and method devoid of the above limitations.

### SUMMARY OF THE INVENTION

According to one aspect of the present invention there is provided a device for modifying an eating behavior of a subject comprising a collapsible device body connected to an elastic tether designed for anchoring through a tissue of a stomach.

According to still further features in the described preferred embodiments the tether is elastic.

According to still further features in the described preferred embodiments the tether does not penetrate completely through a wall of a stomach.

According to still further features in the described preferred embodiments the tether and/or anchoring are selected for enabling the device body to at least partially reside within the duodenum.

According to still further features in the described preferred embodiments the tether and/or anchoring are selected for enabling the device body to shuttle between an antrum and a duodenum.

According to still further features in the described preferred embodiments the device body and/or the tether are designed capable of retaining chyme and optionally transporting a portion of the chyme.

According to still further features in the described preferred embodiments the device body is collapsible under radial forces so as to minimize resistance thereof to a peristaltic wave.

According to still further features in the described preferred embodiments the device body assumes an expanded state during low pressure gastric emptying events in order to facilitate retention of the chyme.

According to further features in preferred embodiments of the invention described below, all portions of the device are sized and configured so as to harmlessly pass through the GI tract when detached from their anchoring site.

According to still further features in the described preferred embodiments the tissue of the stomach is a body of the stomach, the antrum or the pylorus.

According to still further features in the described preferred embodiments an end of the tether is designed for anchoring in or through the tissue of the antrum or the pylorus.

According to still further features in the described preferred embodiments the tether is designed for anchoring in or through the tissue of the stomach.

According to still further features in the described preferred embodiments the tether is sized and configured such that at least some portion of the device body is capable of moving between the antrum and the duodenum when the tether is attached to the tissue of the stomach.

According to still further features in the described preferred embodiments at least some portion of the device body is cylindrical, e.g. egg shaped.

According to still further features in the described preferred embodiments at least some portion of the device body is disk shaped.

According to still further features in the described preferred embodiments at least some portion of the device body is cone shaped.

According to still further features in the described preferred embodiments the device body includes multiple cylindrical, disk or conical elements stacked along a common tether or independently tethered.

According to still further features in the described preferred embodiments the device body is less than 1 cm³ in displaced volume.

According to still further features in the described preferred embodiments the device body is greater than 1 cm³ in contained volume.

According to still further features in the described preferred embodiments a surface area of the device body is less than 50 cm².

According to still further features in the described preferred embodiments the tether is attachable through the tissue via a t-bar anchor.

According to still further features in the described preferred embodiments the device further includes a washer element for preventing erosion of the t-bar anchor into the tissue of the stomach.

According to still further features in the described preferred embodiments the tether is an elastic tether.

According to yet another aspect of the present invention there is provided an implantable device comprising a device body attached to at least one tissue anchor, the tissue anchor comprising a tissue anchoring element attached to a tether having elastic properties.

According to still another aspect of the present invention there is provided a device for modifying an eating behavior of a subject comprising a device body attachable to a tissue of a GI tract, the device body being capable of intermittently contacting a wall region of a duodenum, a pylorus and/or an antrum when attached to the tissue of the GI tract.

According to still another aspect of the present invention there is provided a method of inducing early satiety in a subject comprising attaching a device to a tissue of the antrum or pylorus of a subject in need, the device being configured so as to intermittently contact a wall region of a duodenum and/or the pylorus and/or the antrum.

According to still another aspect of the present invention there is provided a system for modifying an eating behavior of a subject comprising: (a) a delivery apparatus being capable of anchoring a tether in or through GI tract tissue; and (b) a device including a device body attached to the tether, the device being capable of altering the eating behavior of the subject when anchored to the GI tract tissue.

According to still further features in the described preferred embodiments the apparatus includes a vacuum chamber for suctioning a volume of the GI tract tissue.

According to still further features in the described preferred embodiments the apparatus further includes a tissue penetrating element capable of penetrating in or through the volume of the GI tract tissue.

The present invention successfully addresses the shortcomings of the presently known configurations by providing devices and methods which can be used to effectively alter an eating behavior of a subject using a safe, minimally invasive procedure.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 schematically illustrates the stomach-duodenum junction showing the antrum (A), pyloric antrum (PA), the pyloric canal (PC), the duodenum (D), the pyloric sphincter (PS), the submucosal (SM), mucosal (MC), muscle (mu) and serosa (SE) layers and the Pyloric opening (PO).

FIGS. 2A-C illustrate schematically an apparatus for positioning and delivering one embodiment of a device constructed in accordance with the teachings of the present invention.

FIGS. 3A-C illustrate schematically an apparatus for positioning and delivering an additional embodiment of a device constructed in accordance with the teachings of the present invention.

FIGS. 4A-B illustrate two embodiments of the device of the present invention.

FIGS. 5A-C illustrate in detail the system which comprises a device and an apparatus for delivering and positioning the device constructed in accordance with the teachings of the present invention.

FIGS. 6A-B illustrate anchoring of several devices of the present invention in the stomach of a pig and an ex-vivo section of a pig stomach showing that anchoring the device of the present invention using the present approach does not result in any tissue erosion or inflammation despite the fact that it penetrates tissue of the GI tract.

FIGS. 7A-F illustrate the device of the present invention interacting with the pylorus region of a human stomach.

FIGS. 8A-B illustrates in detail a second embodiment of the device of the present invention including key features and dimensions.

FIGS. 9A-B illustrate an apparatus for positioning and delivering one embodiment of a device constructed in accordance with the teachings of the present invention, including the operator's endoscopic view of the delivery device.

FIGS. 10A-D illustrate in detail the system which comprises a device and an apparatus for delivering and positioning the device constructed in accordance with the teachings of the present invention.

FIGS. 11A-D illustrate anchoring of a device of the present invention in the stomach of a pig, interaction of the device with the pylorus region, and showing that anchoring the device of the present invention using the present approach does not result in any tissue erosion or inflammation.

FIGS. 12A-C illustrate an additional embodiment of the device of the present invention.

FIGS. 13A-C illustrate schematically an apparatus for positioning and delivering one embodiment of a device constructed in accordance with the teachings of the present invention.

FIGS. 14A-B illustrate an additional embodiment of the device of the present invention.

FIGS. 15A-E illustrate an additional embodiment of the device of the present invention.

FIGS. 16A-C illustrate an additional embodiment of the device of the present invention.

FIGS. 17A-C illustrate an additional embodiment of the device of the present invention.

FIG. 18 illustrates an ex-vivo section of a pig duodenum showing the effect of a solid device body in the duodenum.

FIGS. 19A-B illustrate a system including one embodiment of the present device and a delivery device for delivering and positioning the device in a stomach of a subject.

FIGS. 20A-C illustrate the effect of a collapsible device body in intact (FIG. 20A) and excised (FIGS. 20B-C) antrum and duodenum of a pig.

### DESCRIPTION OF THE PREFERRED EMBODIMENTS

The present invention is of devices and methods which can be used to alter an eating behavior of a subject.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

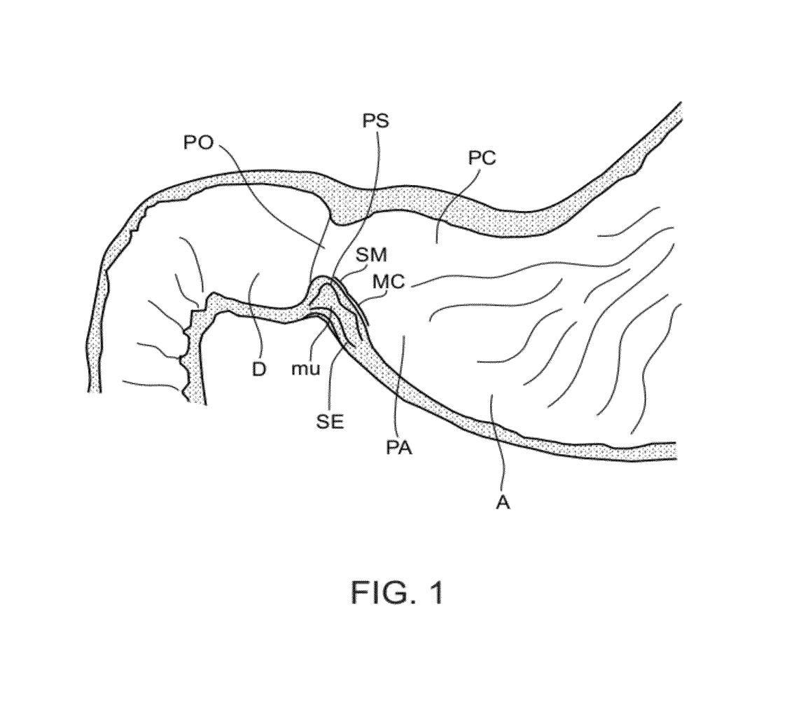

The pylorus is the region of the stomach that connects to the duodenum (FIG. 1). It is divided into two parts: the distal antrum (A) or equivalently the pyloric antrum (PA, FIG. 1), which connects to the body of the stomach, and the pyloric canal (PC, FIG. 1), which connects to the duodenum (D, FIG. 1). The pyloric sphincter (PS, FIG. 1), or valve, is a ring of smooth muscle (mu, FIG. 1) at the end of the pyloric canal which is surrounded by the submucosal (SM, FIG. 1) and mucosal (MC, FIG. 1) layers of the GI tract. The pyloric sphincter is part of a system responsible for controlling the flow of food from the stomach to the duodenum. The pyloric opening (PO, FIG. 1) is the opening surrounded by the lips of the pyloric sphincter (PS) and under certain circumstances also includes part of the pyloric canal (PC). Its diameter varies depending mainly on the degree of contraction and relaxation of the pyloric canal and sphincter. Studies have shown that when open, the diameter of the opening can vary between 5-25 mm.

Anchoring of devices within body tissues, and in particular luminal tissue of body organs such as the stomach presents a formidable challenge. Although several approaches for in-tissue anchoring exist in the art, such approaches are limited by the strength of anchoring, impact on anchor sites within tissues, erosion of the anchoring element through the tissue leading to anchor loss, erosion of the mucosal surface by the device being anchored or by the anchor itself, or the complexity of the anchoring procedure. Anchoring within the stomach with an object larger than can pass though the pylorus results in erosions in the stomach mucosal lining given the constant churning in the antral and body of the stomach. Furthermore, anchoring to dynamic and sensitive GI tissue using barbs or other elements that apply radial forces to the tissue has been documented to cause tissue erosion and inflammation (Rodriguez-Grunert L., et. al, Surgery for Obesity and Related Diseases 4, 2008, 55-59) and therefore is not considered a practical long-term anchoring scheme. In general, it is preferred to anchor in stomach tissue which is stronger, and where a perforation or a partial penetration is less serious than a perforation or partial penetration of the delicate small intestines, which could be life threatening.

While reducing the present invention to practice, the present inventor has devised a device for altering eating behavior and an anchoring approach which facilitates placement of the device body in a lumen of an organ and is both rapid and easy to perform. As shown in the Examples section below, the present device successfully alters a subject's eating behavior while the anchoring approach enables reliable long term anchoring of the device body to tissue of the organ without damaging the tissue. The present anchoring approach is designed for restraining the device body from moving more than a set distance from the anchoring site in the antegrade (or equivalently the aboral or caudal) direction as well as the retrograde (or equivalently the oral or orad) direction, and thus facilitates alteration of the subject's eating behavior.

Thus, according to one aspect of the present invention there is provided a device for altering eating behavior of a subject and a method of anchoring the device body within tissue of a body. Preferably, the tissue is wall tissue of a hollow body organ (e.g. stomach) and anchoring is effected such that the device body resides within the lumen of the organ at a set maximum radius from the anchoring site in any direction.

One preferred use of the present invention is in anchoring devices in the stomach for the purpose of modifying an eating behavior of a subject.

The stomach plays an important role in the digestion of food by chemically/enzymatically breaking down food particles via secreted gastric acid and pepsin and by mechanically breaking down food particles via peristaltic contractions.

In normal digestion, as the stomach fills, digestive glands in the corpus and fundus release hydrochloric acid, a strong acid that helps digest food and facilitate the conversion of digestive enzymes into their active form. The peristaltic contractions of muscles within the stomach wall, especially in the antral region, mix digestive juices and food to produce a semi-fluid substance known as chyme. Chyme is mobilized into the duodenum through coordinated pressure differentials between the antral-pylorus-duodenal regions which are triggered, among other factors, by the consistency of the chyme.

As observed by the inventor, peristaltic waves occasionally start from the pylorus and travel 6-10 cm in the oral direction (retrograde); more commonly such waves start at the antrum and travel in the aboral direction (antegrade). Such peristaltic waves apply forces to antral-tethered devices away from or towards the pylorus respectively. It has been noted in human experiments conducted by the inventor that the retrograde contraction waves are much more intense and powerful than the average antegrade contraction wave and propagate in a lumen-occluding fashion up to distance of 6-10 cm orad from the pyloric opening, and therefore act to either grind food in the distal antrum or push solid objects away from the pylorus.

Furthermore, it has been observed by the inventor using endoscopic means that unrestrained large solid objects that are not easily crushable are freely retropropulsed back into the body of the stomach by the powerful retrograde contraction waves, to a distance of 8-12 cm away from the pylorus.

These observations have led the present inventor to postulate that a device body tethered to the distal antrum would be “run over” by the retrograde contraction wave and pushed back into the body of the stomach and away from the intended site of action of the device (the pyloric region and duodenum).

Due to gravity, large objects settle in the lowest part of the antrum which acts like a sump. When such objects are properly positioned close to the pylorus during an antegrade antral contraction wave, or during an infrequent “lumen-obliterating” phase III MMC wave, they are passed through the pylorus. However, such objects are passed through only after the stomach has been emptied of most of the ingested food.

For example, a US quarter coin 24 mm in diameter and 1.75 mm thick successfully passes through the pylorus of most adults by phase III of the MMC, this is in spite of the fact that regular antegrade peristaltic waves result in a lumen with a smaller diameter as the wave propagate closer to the pylorus. Under such waves, the lumen diameter is reduced significantly in the last 7 cm or so before the pylorus.

The present inventor observed that the closer the device body is to the pylorus, the higher the chance of it being caught by an antegrade antral contraction wave and passed through to the duodenum. Hence it is important to anchor a device body close to the pylorus and if possible (up to 7 cm away), to direct it into the duodenum with a sinker line (further described hereinunder) and/or to make the device body buoyant so that it floats in the antral space near the pylorus; this antral space is situated above the antrum when a person is upright. By anchoring the device close to the pylorus and having a part of the device reside in the duodenum and/or making it buoyant, the device of the present invention should interact or be passed through the pylorus quicker than an unrestrained object of similar size in the stomach. Such an anchoring configuration would substantially enhance the ability of a device body to interfere with gastric emptying.

The functionality of the presently proposed device configuration and anchoring scheme finds further support in the counter-intuitive observation that the pylorus sphincter may not be the main element controlling gastric emptying as the pressure differences necessary to produce antegrade pyloric flow are too small to be measurable by manometry. Flow or chyme through an open pylorus can be caused by a very slight pressure difference between the stomach and duodenum created by highly controlled and coordinated muscle tone differences between the fundus and duodenum (Pal A, et. al. Proc R Soc Lond B Biol Sci 2004; 271: 2587-94, and Pallotta N, et. al. Am J Gastroenterol 1998; 93:2513-22). In fact, approximately one third of emptying sequences occur without gastric peristalsis and with very slight transpyloric pressure gradients (0.15 kPa, 0.6 inch H2O or 0.02 PSI). Hence, it stands to reason that the stomach may empty fairly large volumes of liquids through tone contractions and against minimal pyloric resistance (Ramkumar, D et. al., Neurogastroenterol Motil (2005) 17 Suppl. 1, 22-30). Therefore, it is postulated herein that a device having a properly sized and shaped yet relatively flimsy device body in the region of the pyloric channel and duodenum would effectively resist the low-pressure antegrade flow of chyme and therefore slow gastric emptying.

In addition, premature contractions of the duodenal bulb and the proximal duodenum occur in up to half of all gastric contraction cycles and lead to short spurts of retrograde flow through the pylorus (Hausken T, et. al., Gastroenterology 1992; 102: 1583-90), which should act to return any antrum-anchored device into the antrum temporarily until the next antegrade contraction waves carries it back into the duodenum. Therefore, it is important that the device body is capable of shuttling back and forth to within a radius of 1-15 cm, preferably 3-8 cm of the pylorus and interfere with bulk flow emptying of the gastric content into the duodenum, refluxed bile into the stomach and also retrograde flow in the antrum of gastric contents back into the stomach body.

Pyloric flow is characterized by a sequence of emptying—reflux—emptying cycles. Duodenogastric reflux occurs just before pyloric closure and hence for much shorter episodes than gastroduodenal flow. Reflux was prompted by duodenal contractions, regardless of a propagating or non-propagating pattern. Blocking reflux of bile into the stomach could make digestion less efficient and therefore alter eating behavior.

The human small intestine can generate approximately 20-50 inches of water of peristaltic pressure. It is assumed that the stomach empties at much lower pressures (at around 0.6 inches of water), far from the maximum pressure the intestinal system is capable of generating. Therefore, to interfere with gastric emptying, restriction of the pyloric opening or GI lumen in general via a relatively small and flimsy device body would be sufficient. In fact, the total force applied against, for example, a 12 mm diameter disk-shaped device body by the 0.15 KPa gastric emptying pressure differential is only 3 to 4 grams. The pressure applied to a 22 mm diameter disk-shaped device body is only 10 to 15 grams. Therefore a device body capable of blocking the pylorus does not need to be stiff or bulky in order to partially block transfer of chyme into the duodenum or flow of chyme within the duodenum in order to delay gastric emptying.

The inventor, through experimentation, has determined that a large or rigid object that is intended to delay gastric emptying has larger forces applied to it by peristalsis and grinding of the GI tract than the forces applied during the antegrade flow of chyme through the lumen. Therefore, in cases where a large and relatively rigid device body is used, the major peristaltic forces are transferred to the anchor via the tether and can cause explantation of the device over weeks or months.

In contrast, when utilizing a device body that is crushable/collapsible to a form factor of less than 1 sq cm cross sectional area with a force of 5 Newtons or less, preferably 2 Newton or less, interference with gastric emptying and antral retrograde flow is still achieved, yet the forces acting on the device during the peristaltic waves are small, and therefore the stresses on the anchoring site are lower and allow long-term and even permanent implantation, especially when coupled with non-traumatic elastic anchoring and tethering.

In addition, if the device interferes with the grinding of the food in the antrum (e.g. by partially blocking the retrograde peristaltic waves that mix and grind food) or if the pylorus has to open more widely in order to allow the passage of some chyme through the pylorus due to the partial obstruction of the device described herein, then larger unground particles will be able to pass through a more patent pylorus. Larger particles of food entering the duodenum lead to malabsorption since nutrients such as fat are more poorly extracted out of food particles 0.5 mm and larger (<50% efficiency versus 85% efficiency for sub 0.5 mm particles—Schulze K., Neurogastroenterol Motil 2006, 18, 172-183). Furthermore, the contained or displaced volume of the device body in the pyloric channel can directly offset the volume of chyme available for emptying into the duodenum. For example, a 2-3 ml volume of a device body (whether solid or hollow) in the pyloric channel displaces chyme volume available for emptying into duodenum, which is done in 2-3 ml aliquots per emptying cycle (Schulze K., Neurogastroenterol Motil 2006, 18, 172-183). All of these effects would also lead to a change in eating behavior and a resultant loss of weight.

Without being bound to any particular theory, one mechanism of action of the device of the present invention is a delay of gastric emptying by partial obstruction of the pylorus or pyloric channel. Assuming that a blocking disk of 12 mm diameter resting on a stem of 2 mm diameter is positioned in front of a patent pylorus, then the percent blocking (eclipsing) of a patent pylorus of 7, 9, 11 and 13 mm diameter by the blocking disk would be 100%, 80%, 60% and 40% respectively. Given that the pyloric opening of the average resting pylorus is 8.7 mm in diameter with a range of 5-13 mm (Keet, A. D., The Pyloric Sphincteric Cylinder in Health and Disease), then a 12 mm diameter blocking element is calculated to block approximately 80% of the open area of the average pyloric opening and therefore at least 80% of the flow through the average pylorus by creation of back pressure or a pressure differential between the stomach and the duodenum. Given that the seal is imperfect (the blocking element can move back and forth a bit), then the blockage of flow is probably less than 80%, but still sufficient to cause a noticeable reduction in gastric emptying rates.

In an alternative scenario, video fluoroscopy studies have shown that the pylorus can open to a maximum aperture of 25 mm during gastric emptying, which is roughly the diameter of the small intestines. In such instances, the pylorus stops being a resistor to flow. Therefore, a disk or umbrella shaped device body that is 22 mm in diameter positioned in the region of the pylorus, duodenal bulb or proximal duodenum would effectively block 75% of the outflow and cause a substantial reduction in the rate of gastric emptying and increase satiety.

Physiological reflexes in the form of electrical, hormonal, chemical or muscular signals such as stretch receptors are initiated from the stomach when filled with fluids and by the duodenum in response to the presence of an excess or change in the composition or characteristics of chyme. Such signals are relayed back to other regions of the GI tract (e.g. pylorus and antrum) to slow or even stop food churning and/or stomach emptying; in addition, satiety-inducing (hormonal or electrical) signals are relayed to the brain (Guyton and Hall Textbook of Medical Physiology, pages 785-6; 2006). An example of such feedback is the “duodenal brake” (Schulze-Delrieu et. al., Gastroenterology, 1996; 110:740-747). The duodenum is capable of tonically contracting to mechanically stop or slow transpyloric outflow of contents of the stomach in response to the volume and chemical composition of the chyme. Furthermore, the duodenum can slow gastric emptying by relaxing the gastric fundus, suppressing antral motility, increasing the tone of the pylorus and switching the motility in the duodenum from a propulsive to a mixing pattern.

Thus, and without being bound to any theory, and assuming that the antro-pyloric-duodenal region of the stomach plays an important role in digestion and feedback and proper stimulation, partial blocking or retention of chyme in any of these regions can cause an alteration in eating behavior.

Thus, according to one aspect of the present invention there is provided a device for modifying an eating behavior of a subject. As used herein, the term subject denotes an animal, preferably a mammal such as a human, e.g. a human having an eating disorder or a weight related disorder such as obesity.

One embodiment of the present device includes a device body which is anchored to GI tract tissue in a manner which enables intermittent contact between the device body and mucosal tissue residing within the antrum, pylorus and proximal region of the duodenum (e.g. duodenal bulb), as well as the duodenal side of the pylorus. In contrast to prior art devices, the device body of the present invention is not fixed in one location in the GI tract, nor is it free floating in the stomach, but rather it is allowed to move axially in both directions relative to the GI tract against the restraining effect of an anchored tether of a defined length around a selected anchoring point. Such an anchoring scheme can be used to alter eating behavior, by for example stimulating early satiety feedback.

This embodiment of the present device can also be used to create partial and/or temporary obstruction at the pylorus or the pyloric channel. Numerous reports in the medical literature report the physiological outcomes resulting from partial gastric outlet obstruction at the pylorus. Partial gastric outlet obstruction can result from the presence of a sessile or penduculated gastric polyp 1-3 cm in diameter, which partially obstructs the free movement of digested food from the stomach through the pyloric channel and/or the pylorus itself and into the duodenum. The physiological signs associated with such partial gastric outlet obstruction are primarily early satiety, mild-to-moderate nausea and to some degree, loss of appetite and weight. Thus, the positioning of the device of the present invention in the immediate vicinity of the pylorus as described in the Examples below, enables it to partially limit the amount of digested food passing through the pylorus, and through such controlled interference, imitate the effects of a polyp in altering eating behavior.

A presently preferred embodiment of the present device includes a device body which is directly or indirectly attachable to tissue of an antrum or a pylorus in a way which enables the device body to intermittently contact the pyloric region when attached to the tissue of the antrum or the pylorus. As used herein, the phrase “antral-duodenal region” denotes any mucosal tissue residing within a region of the gastrointestinal (GI) tract starting at the proximal antrum and terminating at the distal duodenum including the pylorus and pyloric regions of the stomach.

It will be appreciated that although antrum or pyloric anchoring is presently preferred, other GI tract anchoring sites (e.g. body of the stomach, fundus, lower esophageal sphincter, nasal cavity etc) are also envisaged with the tissue-penetrating means and tether length selected appropriately.

In an alternative embodiment, the present device includes more than one device body (e.g. 2, 3, 4, 5, 6 or more), such device bodies (which can be identical or of different sizes and/or shapes) can be placed at intervals on a tether the entire length of the duodenum (˜26 cm), jejunum (˜2.5 m), ileum (˜3.5 m) or any combination of the three to effectively alter the flow of chyme throughout any and all portions of the small intestines. Preferably, the device body or bodies of the present invention are anchored to tissue in a manner which enables intermittent contact between the device body or bodies and mucosal tissue residing within the antrum, pylorus and proximal region of the duodenum, duodenal bulb, as well as the duodenal side of the pylorus. To that effect, anchoring of the device bodies to tissue is preferably effected via a tether.

The tether can be any elongated element constructed of any biocompatible material. Examples of suitable tether include suture material, elastic or inelastic, braided or monofilament sutures, wires, ribbons or strings (e.g. made of a metal, a polymer, elastomer, natural materials, or combinations thereof).

The device body can be of any shape or size depending on the site of implantation and function. The device body can be delivered in a compacted or collapsed state and expand into a deployed state slowly over a period of time following implantation to allow the patient to get used to the device gradually. For example, a device body in the form of a normally-open elastically-compressible parachute or umbrella can be adhered closed with a water-dissolvable film or glue that dissolves gradually after implantation and allows the device body to assume it's naturally open parachute-like shape over a week or two in order to not create too large of a shock to the patient if the open device body suddenly slows the gastric emptying rate by 75%. Alternatively, the device body can be expanded using fluid or gas introduced via a filling tube or port (e.g. septum) to vary the shape, volume, rigidity, or outer diameter of the device body post implantation to allow for a patient to gradually learn to live with the device, or to adjust the size or efficacy of the device in either direction dynamically, or periodically much like an adjustable lap band. Control of such volume or shape changes can be via remote control from outside the patient's body.

Furthermore, the length of the tether, the position of device body on the tether or the size of the device body can be fixed, or controlled during or following the anchoring procedure by the user/doctor or automatically via a physiological parameter (e.g. presence of food, slow expansion due to osmosis-drive fluid inflow) to enable accommodation or adjustment of device position or function.

In addition, the tether or device body can be configured to provide an indication of a release of the tether from the tissue or from the device body. For example, if detached, the device body can release an indicator dye so that the patient knows the device is detached (e.g. methylene blue).

The device body can be configured to function as or to carry electrical components (e.g. transceivers, electrodes, cameras, valves, coils, batteries, capacitors, a miniature winch to draw in the tether and move the device body against normal peristaltic flow, etc) and/or compositions (e.g. medicaments), and can be configured to change shape and/or size (e.g. inflate via chemical reactions, compressed gas, osmotic liquid flow) depending on external signal and environment conditions.

For example, the tether can act as an electrode embedded in the mucosal, submucosal, muscle or serosal layers of the stomach using and be used to alter (enhance in the case of gastroparesis, or delay in the case of obesity) gastric motility using electrode positions and stimulation parameters known in the art. Many external gastric stimulators are known in the art. An electrode carrying device of the present invention can be introduced and implanted using an endoscope, which is significantly more tolerable than an open or laparoscopic surgical procedure used in existing gastric electrical stimulators. Furthermore, the tether electrode can be located in proximity to know nerves (vagus etc) that innervate the stomach. The entire tether electrode device can be passive and connected via wired or wireless means to an electrical energy source. The device body in this embodiment can be the size of the anchoring disk (say 1 mm thick by 5 mm diameter) and the tether electrode of approximately 5-25 mm length would therefore have minimal impact in the stomach and survive long term with no adverse effect, much like a single pass of a non-absorbable suture will remain in the stomach for decades. Alternatively, the metal electrode can be a very thin wire (e.g. 50-100 microns in diameter) wrapped spirally around an elastic tether such as a silicone cord (e.g. 0.5-2 mm in diameter) which will behave as a fully elastic tether with electrode properties.

The device described above can run open or closed loop (an example of the latter is that it turns on when a subject is eating and gastric acid is being generated). Furthermore, the device can be configured for remote charging (e.g. coil induction) or for using the acid in the stomach to generate electricity. The device can have an on board battery or capacitor (within the device body) to store excess energy for future use, onboard electronics to regulate and condition the stimulation parameters, interface with sensors that detect parameters such as the current pH, presence and composition of food, as well as wireless communication capabilities to allow for control of the device from outside the body. Such functions can be carried out by a BION-like device (BION—Advanced Bionics inc.) which can be provided with a soft coating (e.g. silicone shore A 70 or less), to enable it to live in the stomach environment without causing tissue erosion. The device body can include a radio-opaque marker such as barium sulfate or metal, or an RFID unit to confirm presence/absence in the GI tract. An active RFID unit can also relay sensor data (e.g. ph, temperature, pressure, etc).

BION-like devices of the present invention can also be implanted along the intestines and be used to pace intestinal contractions (peristalsis) in cases such as neurological deficit induced constipation.

Alternatively, the device body can function as an electrode in which case stimulation is provided upon contact with the wall of the stomach. A BION-like device can be anchored by one end in the antrum to ensure tissue sampling (intermittent tissue contact) while preventing erosion. The anchor or tether can also act as the electrode surface through which electrical current or charge is delivered into the stomach tissue.

A system of several devices (device body and tether) can be implanted at various locations within the stomach and used to coordinate stimulation at various locations in the stomach wall through controlled electrical impulses provided from the device bodies or tethers which can act to generate, inhibit or entrain peristaltic waves. Control of such devices can be effected through a central control unit disposed in one of the device bodies or through an extracorporeal control unit. The various devices can intercommunicate and/or communicate with the central control unit via a wired or a wireless connection.

The device of the present invention can also incorporate several other mechanisms for electrically stimulating tissue. For example, the device can include electrodes positionable on a device body and powered by a power source positioned at the site of tether attachment. Such a power source can be a battery. The tether can include insulated wires for carrying the current produced by the power source to the electrodes positioned on the surface of the device body. Since the device body only intermittently contacts tissue, an electric current will be periodically applied to the tissue.

Electrical stimulation of the GI tract tissue can be effected using electrodes embedded in the mucosal, submucosal or muscle layers of the stomach using endoscopically-introduced electrode anchors or screws and thereby be used to alter (enhance in the case of gastroparesis or delay in the case of obesity) gastric motility using electrode positions and stimulation parameters known in the art. The device described herein can be introduced and implanted using an endoscope, which is significantly more tolerable than an open or laparoscopic surgical procedure used in existing gastric electrical stimulators.

The device body can also be configured to function as a drug depot that releases drug under the control of environmental conditions (e.g. acid release) internal sensors or external command. Such a device body can be used for extended release of medication such as proton pump inhibitors (PPIs) under pH control for example. Alternative drugs that can be released include agents that slow down gastric emptying, such as atropine, gastrin, neurotensin, morphine, sumatriptan, calcium channel blockers or other agents known to slow gastric emptying via various mechanisms. Other types of drugs, such as the hormone CCK, can be released into stomach or small intestines to cause early satiety. The device body can also be configured for reloading once the medication reservoir is spent. Such reloading can be effected in a number of ways (e.g. a hollow fill tube running from the device body which acts as a reservoir up through the esophagus to the nasal cavity, or alternatively magnetic coupling in which the device body includes a magnet and the drug is provided orally in the form of magnetic or paramagnetic particles such as iron particles coated with or containing the drug).

The device of the present invention is anchored to the GI tissue of interest using the techniques described herein, at any point along the GI tract from the mouth to the anus or in the nasopharynx, nasal cavity or nares region.

The device body can also be configured as an inflatable balloon that is inflated post anchoring by a fill tube as above, or by an internally-contained compressed gas source to take up volume in the stomach and induce satiety. Alternatively, inflation can be effected via absorption of water from the stomach environment using, for example, an osmotic pump such as that described in U.S. Pat. No. 5,005,591.

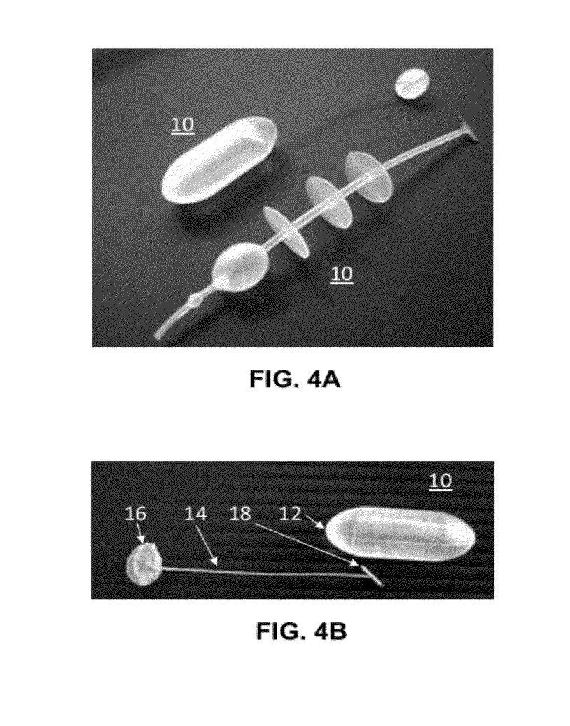

An example of two embodiments of device 10 of the present invention is illustrated in FIG. 4a. Device body 12 in FIG. 4b which is configured for implantation within a stomach lumen is shaped as a hollow cylinder with tapering ends. The Examples section which follows provides further description of this device body and its use.

In order to be capable of attachment to both tissue and the device body, the tether includes two anchoring/attachment ends. A first end is configured for anchoring the tether to the tissue via partial or complete penetration of the tissue, and a second end is configured for attaching the tether to the device body.

Attachment of the tether to the tissue can be effected via various anchoring configurations which rely upon penetration and positioning of the anchor against tissue (e.g. external surface of a wall of the organ, or against a tissue fold) at the site through which the tether is inserted. Such anchoring can be provided by t-bar, flat disc, ball, basket, hook or coil configurations of the first end of the tether. The end of the tether can be designed to be of a shape and size small enough to burrow in the tissue of the organ due to axial forces present on the device body, or large enough to distribute such forces over a large enough area to prevent burrowing of the anchoring end of the tether into the organ tissue. In a further embodiment, the end of the tether not pushed through the tissue can be attached to one or more device bodies as well, thereby anchoring more than one device body in the same area of the hollow organ with one tether in a “dumbbell” configuration. The device bodies themselves can act to limit the back and forth motion of the tether through the tissue.

Attachment of the tether to the device body can be effected via any one of several approaches capable of maintaining a secure connection between the tether and device body while being amenable to in-body attachment.

Such anchoring can be effected via various couplers such as hook and loop configurations wherein the tether is provided with a hook and the device body with a loop (or vice versa), via magnetic attachment, deployment of a t-bar, hook and the like.

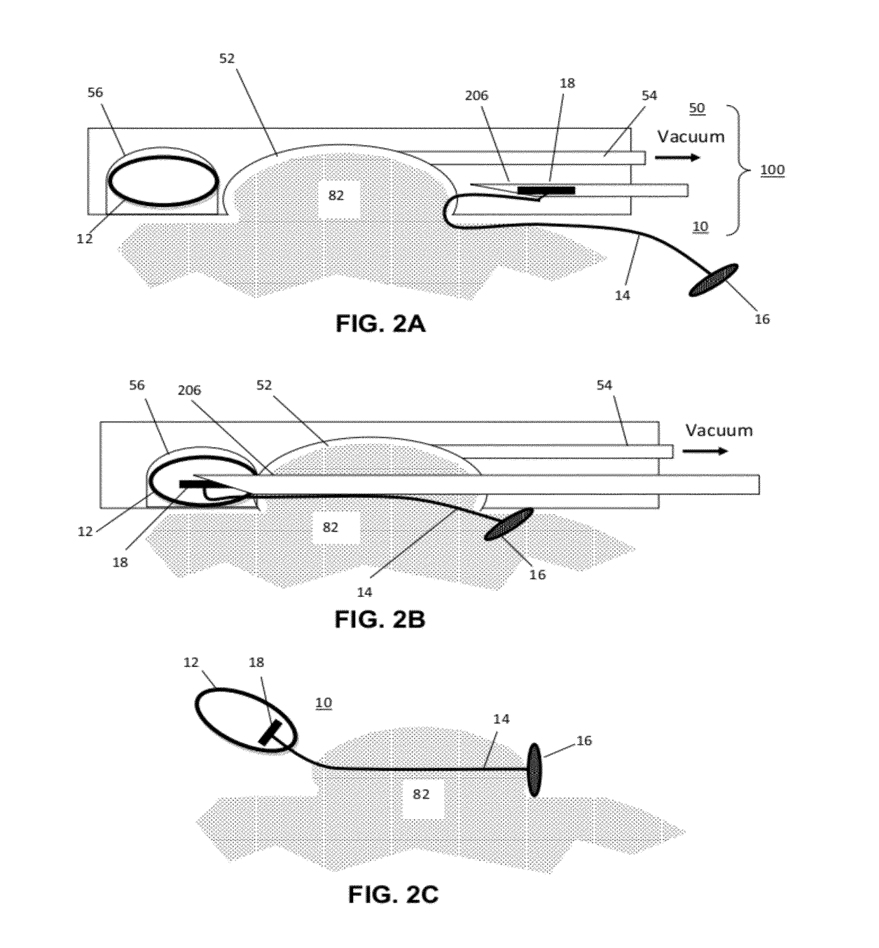

An example of a preferred tether anchoring configuration is shown in FIGS. 2a-c. In this case, tether 14 is provided at the first end with backstop element 16 which is positioned against the tissue at the site of tether insertion. The second end of tether 14 is fitted with a t-bar anchoring element 18 which can be pushed via a guide (e.g. slotted needle) into the device body (in this case, a hollow space within the device body). An alternative to a t-bar would be a Nitinol element that is preformed into a pig tail or flat spiral shape and for delivery is forced straight inside a delivery needle. Such anchoring is advantageous since it provide secure attachment while sequestering anchoring element 18 within device body 12 thus effectively shielding the tissue from anchor element 18 and potential tissue damage which can be caused by its edges. In fact, even though the anchor element 18 may be made of metal, the only materials in contact with the tissue in the present invention as described in the FIGS. 2a-c are those of device body 12 (e.g. silicone), tether 14 and backstop element 16 (e.g. silicone, polypropylene, polyurethane, polyester or the like) ensuring long-term biocompatibility and minimizing the erosive effects of hard materials such as metal rubbing against a sensitive and dynamic mucosal tissue surface.

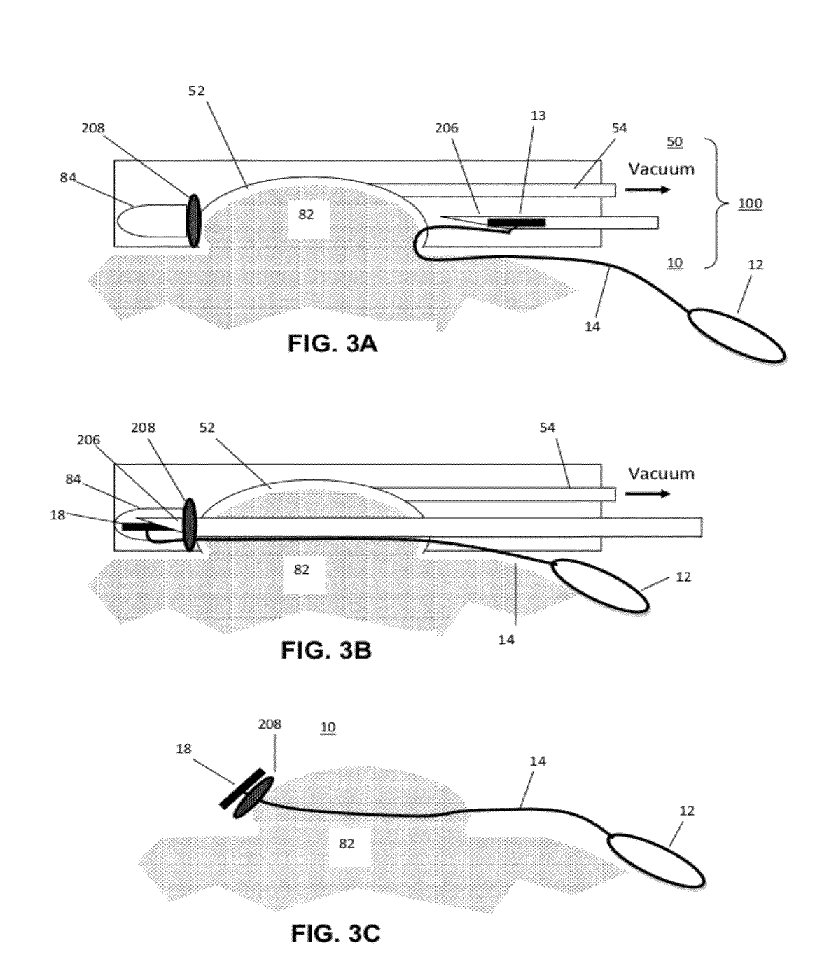

When used to anchor device body 12 within a lumen of an organ, tissue anchoring is effected as through-tissue anchoring. Through-tissue anchoring involves having the tissue anchor side of the tether reside outside the lumen of a body organ (delivered transluminally or transmurally and resting outside the stomach and against the serosa with or without a washer element) or alternatively inside the lumen anchored through a tissue fold (as shown in FIGS. 2 and 3).

Several approaches can be used to effect such anchoring. Delivery of the tether through the wall of the hollow organ and into the lumen can be effected by endoscopic, percutaneous, laparoscopic or open surgical delivery alone or combined with a procedure where the device body is delivered endoscopically. Delivery through a tissue fold can be effected via a fully endoscopic procedure. In any case, anchoring of the tether to the tissue and the device body may necessitate alignment of the device body with the tissue and second end of the tether in a “forward anchoring” configuration where the device ends up positioned in front of the first needle penetration point. An endoscopic approach for anchoring is further described below and in the Examples section which follows.

FIGS. 4a-b illustrate two embodiments of the device which are both referred to herein as device 10. In FIG. 4b, device 10 includes a device body 12 which is shaped as a hollow cylinder with closed tapering ends. Device body 12 is 25 mm long and 10 mm in diameter and hollow with an average wall thickness of 2 mm and is fabricated from silicone or another biocompatible and acid/chyme resistant material using molding techniques. Device body 12 can include various configurations ranging from a single object to bunched objects (likes grapes) and surfaces that are smooth, pitted, bumpy, filamentous, etc. Device body 12 can be open ended or sealed, spherical or tube shaped. Device 10 further includes an attachable tether 14 which includes a suture thread (e.g. 2-0 monofilament polypropylene suture or a 0.3-3 mm diameter silicone or polyurethane cord) provided with a backstop element 16 and an anchoring element 18. Backstop element 16 includes a flat disc which is constructed from silicone (or any other shape and material suitable for providing a 1-25 sq mm of anchoring surface against tissue in order to minimize burrowing or erosion thereof), while anchoring element 18 includes a t-bar element which is fully or partially constructed from a metal (e.g. stainless steel, titanium, Nitinol) or a hard polymer. Depending on the site of anchoring, tether 14 can be 0.5-10 cm in length. Preferably, the length of tether 14 is selected such that following anchoring of device 10, the distance between the tissue and device body 12 is at least 0.5 cm. This ensures that device body 12 operates within a defined region within the stomach (e.g. confined to the pyloric channel) yet also has enough freedom of motion to not constantly contact a single area of the tissue which could lead to tissue erosion. It has been noted experimentally by the inventor that such erosion occurs if the device is anchored so that device body sits directly on the tissue with too short of a tether. In cases where the exact location of the device is not critical, the distance between the tissue and device body 12 can be greater than 2 cm. In cases where the tether is elastic, even less of it is necessary to be exposed as any forces in the hollow organ will tend to pull the device temporarily and harmlessly away from its anchoring point. Furthermore, single point anchoring limits the forces on the device body to one direction only—axial relative to the tether. If more than one tether or anchoring point is used, relative motion of the anchoring points can put significant stresses on the device body, causing tearing of the tissue by the tethers which may be pulled off axis by the resultant force vectors, as well as erosion and/or ulceration of the tissue.

Device 10 includes at least one device body 12 and one tether 14. When device 10 is in use, backstop element 16 of tether 14 rests against a tissue surface. Backstop element 16 can be formed via a second device body 12, a loop of the tether material itself, a t-bar anchor, coil, suture thread, staples, clips, disks, washers and the like.

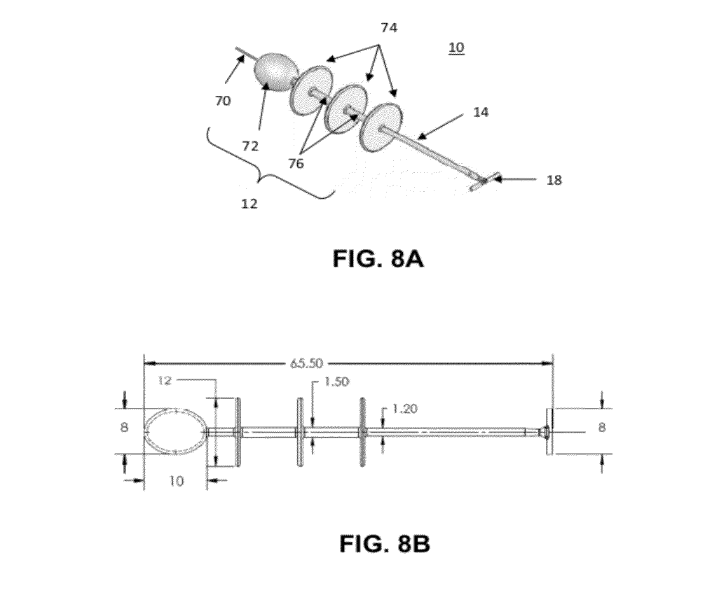

FIG. 8a-b depicts an additional embodiment of device 10 with key features indicated in FIG. 8a and typical dimensions (in mm) of device 10 indicated in FIG. 8b. With reference to FIG. 8a-b, device body 12 comprises front tether 70, front ball 72, disks 74 and stems 76. Front tether 70 allows temporary attachment of the front portion of device 10 to endoscope 68 or needle guide 58 to keep device 10 pointing backwards in the orad direction upon intubation of a patient with system 100. Front tether 70 can also be used as part of a “sinker line” that continues into the small intestine to keep device 10 pointing in a caudal direction or running through the pylorus in the resting state as described later.

Front ball 72 serves to move the device along with the peristaltic wave into and out of the duodenum. Front ball 72 also acts to partially block pyloric opening 80. Front ball 72 is retained in the duodenum to position disks in front of or immediately behind pylorus. Front ball 72 also serves as an object to stimulate antral, pyloric and duodenal mechanoreceptors. Front ball 72 can be hollow (e.g. 0.5 mm wall thickness filled with trapped air) which makes it soft enough to prevent surface erosion of the mucosa and buoyant enough to float in a liquid filled antrum and stay in close proximity to the pyloric opening which is in the upward direction in an upright human. Front ball 72 is designed to compress to half of its diameter when a lateral force of 1-10 Newtons, preferably 2-5 Newtons, is applied to it. Such easy deformation of front ball 72 (and also disks 74) will prevent the erosion of the mucosa during friction and grinding of device 10 in the antrum. For this purpose, front ball 72 can also be made from very soft polymer, such as shore A 5 silicone, whether solid or as a closed or open cell foam, that is glued or molded together with a harder polymer such as shore A 60 silicone used for making tether 14 and anchoring element 18.

Stem 76 allows for multiple stable resting states of the device with the stem running through the pylorus. Stem 76 is soft and deformable, yet gives the overall device geometric stability so that disks 74 are kept normal to the axis of stem 76 which helps keep device 10 off the wall of stomach, increasing the chance of device 10 being caught by an antegrade wave and being pushed against or through pyloric opening 80.

One or more disks 74 serve to partially block pylorus opening 80 during antegrade food transport which occurs at very low pressure differentials between the stomach and duodenum. Disk 74 when present in the antrum and facing the orad direction also serves to block the mixing jet during retropropulsion grinding waves. Multiple disks 74 stacked along a common tether 14 or on separate tethers 14, ensure that endoscopic positioning accuracy of device 10 can be in a range of plus or minus a few cm and that at least one disk 74 will always be in proximity to pyloric opening 80 even if device body 12 is shuttling back and forth through the pylorus. Disk 74 can function to block flow whether it is pushed up against a narrow pylorus on the stomach side, or pulled back against the duodenal side of the pylorus by the elasticity of the tether. The portion of disk 74 that obstructs the pyloric opening is cantilevered into the passageway, but also partially supported by the majority of disk 74 that is resting against the surface of the pyloric tissue. Disks 74 can be 5-40 mm in diameter, preferably 12-25 mm in diameter and can be flat, conical or generally concave with or without supporting elements to prevent them from inverting.

Tether 14 serves to elastically anchor device 10 at a distance of 0-20 cm, or preferably 2-10 cm or more preferably 3-5 cm from the pyloric opening and allow sufficient axial motion to let device body 12 escape towards the orad direction the high pressure retrograde antral contraction waves that occur within 0-6 cm of the pylorus. The distal 0.5-3 cm, preferably 1-2 cm, of tether 14 is implanted through the submucosa/muscularis/serosa layer of the stomach using delivery device 50, or anchored completely through the tissue to the outside surface of the organ using a T-anchor or similar.

Anchoring element 18 can be made, for example, as a 0.8 mm outer diameter silicone T anchor overmolded onto a 0.010″-0.020″ diameter stainless steel wire with bar-bell type ends for stiffness. The anchoring element can be made to fit inside a slotted 18 or 19 gage delivery needle.

Device 10, as described above and illustrated in FIG. 8a-b can tolerate at least 2 Newtons, preferably 5 Newtons and more preferably 10 Newtons or more of tensile force along tether 14 before breaking. Under a tensile force of 5 Newtons, an elastic tether 14 increases in length by approximately 100% to 800% depending on the material selection, material hardness and diameter of tether 14.

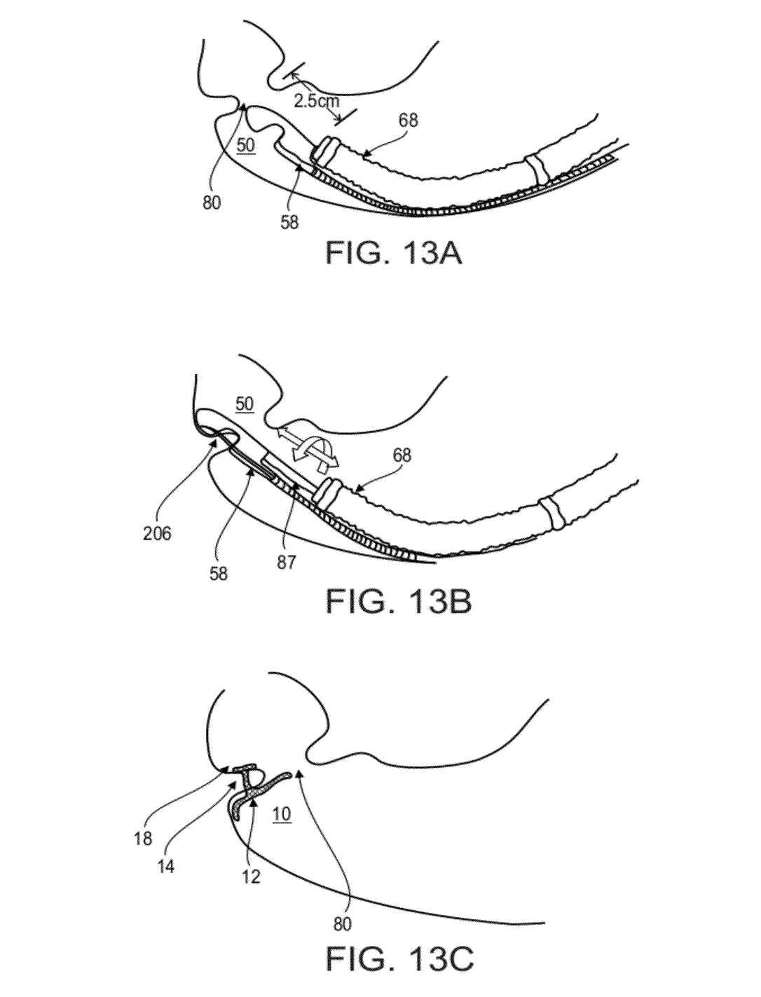

Tissue anchoring is classified herein as through-tissue or in-tissue anchoring. In-tissue anchoring implies that the anchor and part of the tether rests within the tissue (e.g. anchored with an in-tissue coil or barb). Through-tissue anchoring involves having the anchor reside outside the lumen or with the anchor residing inside the lumen and only part of the tether residing in the tissue itself. Through-tissue anchoring is exemplified by FIGS. 2, 3 and 13. Through-tissue anchoring can be effected with a needle using direct visual guidance through the working channel of an endoscope (see FIG. 13). Alternatively, a vacuum cup can assist in through-tissue anchoring (see FIGS. 2 and 3).

Suitable tissue anchors for both in-tissue and through-tissue anchoring can include t-bar or mushroom-like elements which can be buried within the tissue or juxtaposed against the tissue at the exit site. Methods of inserting such anchors include open surgery, laparoscopic or endoscopic means known in the art and developed for such procedures, e.g. natural orifice transgastric endoscopic surgery (NOTES).

In any case, tether 14 is attached to the tissue of the antrum in a way which ensures a secure connection which can withstand the forces acting on device body 12 and tether 14 during GI tract movements. Tether 14 or backstop element 16 can be designed to degrade and detach after a set time in the acidic or bile environment of the stomach or duodenum respectively. In this manner, device 10 can be designed to remove itself after a set time and device 10, or components thereof, can pass harmlessly through the GI tract and be removed from the body.

Device body 12 can be fabricated from a wide range of biocompatible materials. Examples of suitable materials that can be used alone or in combination with one another include polymers such as polyurethane and polypropylene, silicone, latex, PTFE, ePTFE, thermoplastic elastomers such as SBS (polystyrene-polybutadiene-polystyrene or styrene-butylene-styrene), PEF, ceramics, NITINOL, passive metals, alloys and the like.

Additional coatings for preventing biofilm formation, encapsulation, erosion and antigenic reactions can also be employed. The prior art is replete with examples of materials that can be used for such purposes [see for example, Baveja et al. Biomaterials. 2004 September; 25(20):5003-12] or Surfacine™ (www.surfacine.com).

Coatings including medicaments or pharmaceutically active agents are also contemplated herein, examples of active agents include, but are not limited to hormones such as CCK, ghrelin, motilin and the like. Alternatively, coatings which stimulate chemoreceptors (e.g. fat or fat-like substances, sugars and the like) can also be utilized. Non-releasable coatings (e.g. attached through non-degradable linkers) are preferred for prolonged effect.

Furthermore, the device of the present invention can be an endoscopically-refillable reservoir for medicaments, pharmaceutically active agents such as hormones small molecules or other peptides, as well as chemical agents such as, by way of example, hydrochloric acid, which is known to suppress motility when in contact with the duodenal mucosal surfaces.

Device body 12 is selected of a length and diameter so that it can pass in its entirety safely through the GI tract if it becomes detached from the anchoring tissue, thereby minimizing the risk of a blockage of the pylorus or small intestines. In case the static diameter (when in a normally open position) of device body 12 is larger than 25 mm (the largest average size of an object that can pass the pylorus opening), then device body 12 is designed to be very flimsy (e.g. very thin walled hollow ball, cone or a flat and thin silicone disk) so that it can easily deform or collapse to be less than 25 mm diameter and therefore pass through the GI tract if detached from the anchoring tissue.

For a device body in the shape of a solid or thin-walled closed hollow object, the contained volume (defined as the volume of fluid that a device body can contain within it when oriented in the maximal fluid retention configuration) is roughly equal to the displaced volume of the device body (defined as the volume of fluid displaced by a submerged device body). For example, a closed and hollow thin-walled sphere 2 cm in diameter contains and displaces approximately 4 cm³ of volume.

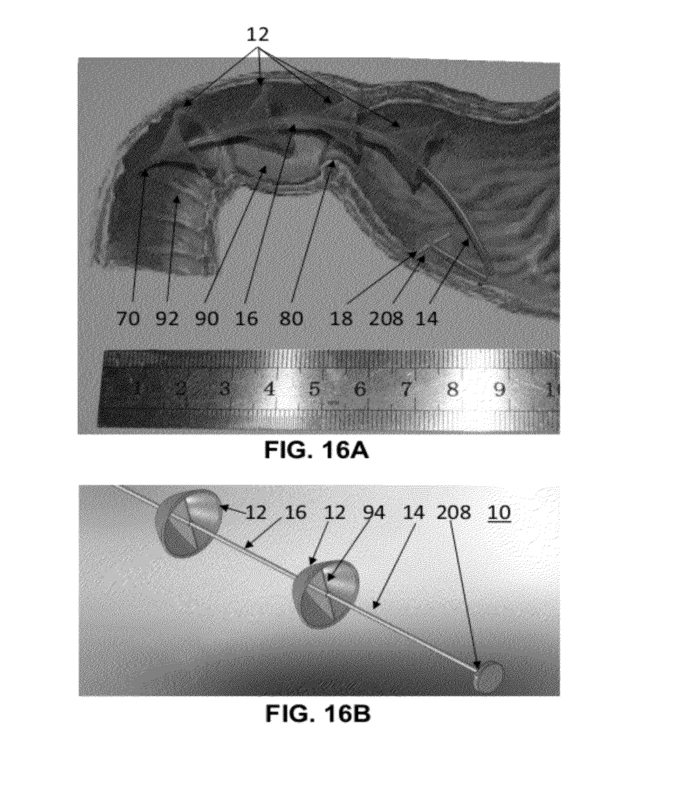

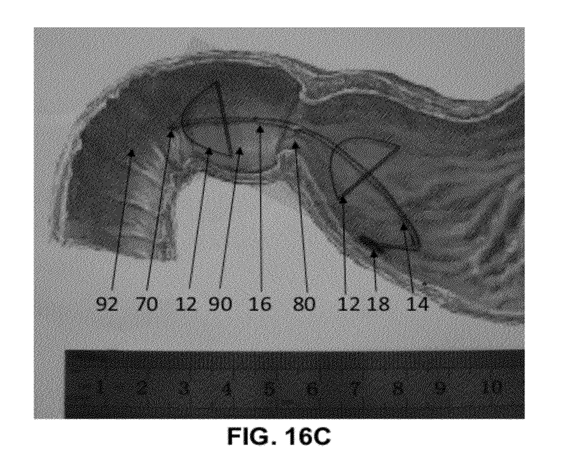

For open structures, however, the contained volume is determined by the volumetric envelope of its outer surface and the displaced volume is simply the surface area multiplied by the thickness of the device body. For example, in the configuration of device body 12 illustrated in FIGS. 16b and 16c, the two hemispherical device open bodies 12 which are 2 cm in diameter can together maximally contain approximately 4 cm³ of fluid. The total surface area of both device bodies is approximately 18 cm² and the thickness of the device body wall is approximately 0.03 cm, leading to a displaced volume of approximately 0.5 cm³. Thus, the design of device body 12 in this configuration can achieve a contained volume of a hollow sphere of the same diameter, yet only have a displaced volume ⅛ that of a hollow sphere. This is important as flow retardation is related to the contained volume, and the forces acting on the device body to explant the tether from the tissue are related to the displaced volume as described in more detail herein.

The contained volume of device body 12 is typically selected from a range of zero cm³ (when collapsed by a contracting lumen) to 30 cm³ (when deployed in its normally open position in a patent lumen). Preferably, the displaced volume of the entire device is less than 4 cm³ to minimize the forces applied to the device during peristaltic waves. Such volume can be distributed over a cylindrical shape, having a length of 1-4 cm and a diameter of 0.1-3 cm. Other shapes contemplated herein include hollow or solid, open or closed spheres, ellipses (e.g. egg or torpedo-shaped), discs, cubes, triangles, cones, umbrellas, protruding fingers, amorphous shapes and the like. The goal of designing device body 12 is to maximize the contained volume which hinders bulk gastric emptying while minimizing the displaced volume which relates to the magnitude of forces device body 12 is subject to during peristalsis, grinding, churning, or endoscopic introduction into the GI tract for implantation.

Various shapes can be connected in parallel or series on one or more tethers 14. The surface of device body 12 is preferably smooth so as to minimize any shear forces applied to mucosal tissue of the antrum, pylorus and/or duodenum and to minimize the chance of bezoar formation around device body 12, but also potentially ridged to better stimulate the tissue. Alternatively, the surface of device body 12 can be porous, pitted or shaped in the form of one or more cups to retain a bit of chyme on the device surface via capillary forces and therefore prolong the stimulation of the chemical sensors in the duodenum that sense the presence of chyme, hence slowing gastric motility.

Device body 12 can be fabricated using any one of several well known fabrication techniques including, but not limited to, casting, transfer or injection molding, extrusion, machining and the like.

A device body 12 fabricated from silicone having a Shore A hardness range of 5-100 is presently preferred for its biocompatibility, durability and low surface hardness. It is preferable that all parts of the device that are in contact with the submucosa of the GI tract are soft enough to bend, deform and extend elastically to a degree sufficient to not cause erosion of the mucosal surfaces despite the pressure and motion exerted on the device by the GI tract.

Device body 12 can be attached directly to the GI tissue via backstop element 16 if excessive movement relative to the anchoring point is not desired. Alternatively, the length and composition of tether 14 is selected according to the intended function of device 10. The length of tether 14 can be anywhere from 0.5 cm to 700 cm and largely depends on the site of attachment of backstop 16 and function of device 10. For example, device body 12 can reach the pylorus if backstop element 16 is attached to the nasal cavity through the esophagus using tether 14 of roughly 50-70 cm length. To reach from the pylorus to the end of the jejunum would require tether 14 to be approximately 300 cm long.

Tether 14 is generally floppy and flimsy and cannot resist any appreciable bending or compressive force and can be non-elastic or elastic; a non-elastic tether 14 can be fabricated from a polymer (e.g. polyethylene, PTFE, polypropylene, or nylon) or metal; while an elastic tether 14 can be fabricated from silicone, polyurethane, rubber, latex, and the like. The elastic tether can have an elastic configuration and yet be made from a non-elastic material, e.g. a coil made from a polymer, or it can be fabricated from an elastic material such as silicone. A tether having a first portion which is elastic and a second portion which is not elastic can also be utilized by the present invention.

As used herein, the phrases “elastic”, “elastic properties” or “elastic compliance” are used interchangeably to refer to the ability of the tether or a portion thereof to reversibly increase in length under a pulling force. Such an increase in length can be at least 10%, preferably at least 25%, more preferably at least 50% before breaking or undergoing plastic deformation. The elastic properties of the tether can be provided by the tether structure, cross sectional and axial geometries and/or tether material.

The tether can be a hollow or solid thread or string-like structure which includes one or several adjoined portions. If hollow, fluids or gases can be transported through the tether to and from device body 12. The tether can be made out of a twisted or braided set of smaller elastic filaments, much like a bungee cord. Such a braided design will allow cellular ingrowth and better integration into the host tissue, A tether constructed from two adjoined portions can be used to provide a unique elastic profile, wherein one portion elastically stretches and another does not, or where both portion stretch, each to a different degree. A multi-portion tether configuration can also be used to simplify construction of the anchor of the present invention. For example, the anchoring element and a first portion of the tether can be molded from a single material and attached to a second and elastic portion of the tether via gluing, press fit, over-molding and the like. A multi-portion tether configuration can also be used in cases where different portion are exposed to different environments, for example, when a first portion of the tether resides within a tissue and another in a lumen. The tether material can be inelastic and yet the tether can be configured to provide elasticity, e.g. an elastic coil structure. For example, the tether can be inelastic and be wound around a rotary-spring-loaded drum in the device body to allow for an elastic effect with inelastic materials.

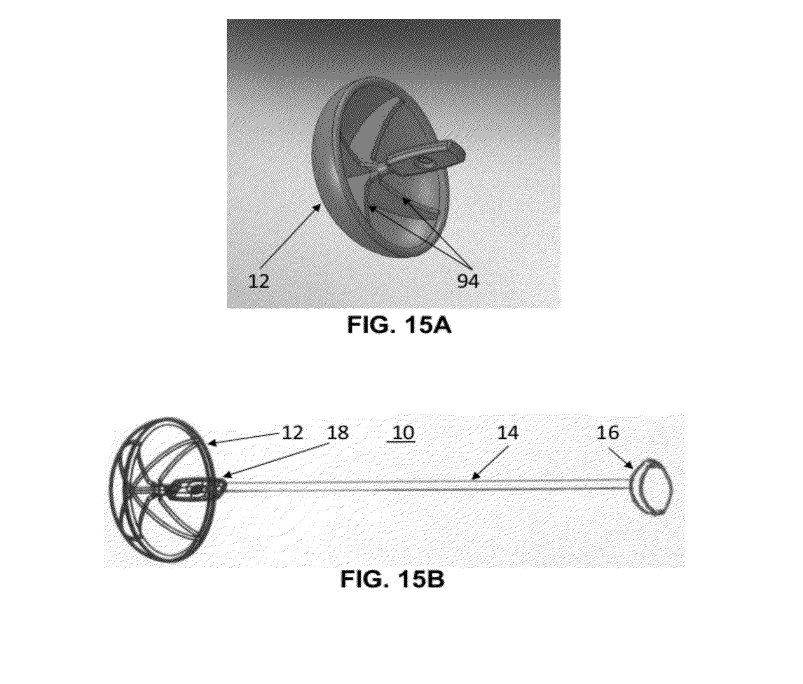

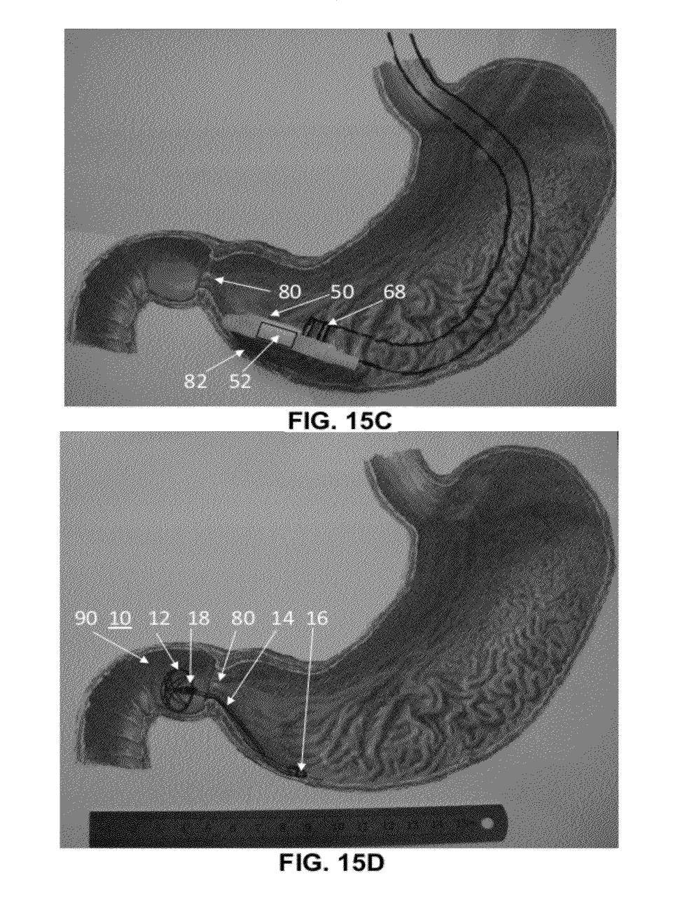

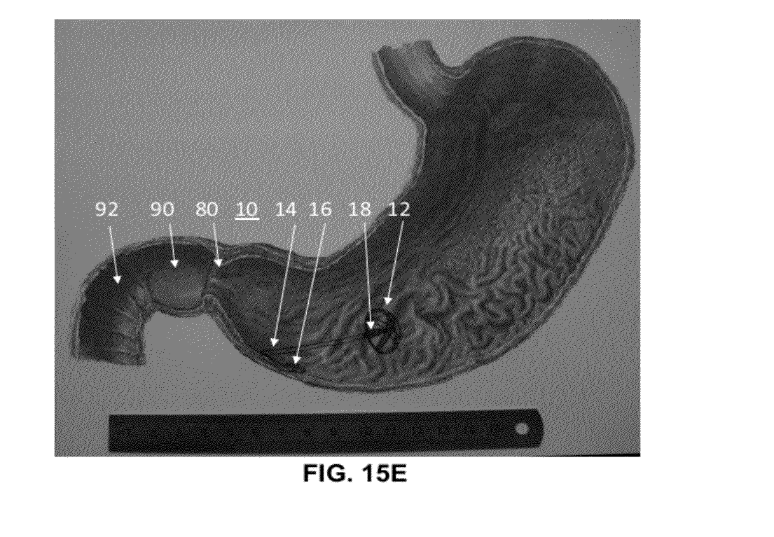

Tether 14 can be made of material having a selected hardness. In general, a softer material is preferable to a harder material to avoid cutting, eroding or remodeling the tissue tract through which the tether runs if forces are applied to the tether over the long term. For example, polypropylene has a durometer hardness of around 60 on the Shore D scale and can cut or erode tissue like a cheese grate when under long term tension leading to eventual detachment of any anchored device (as will be described in the Examples below). However, silicones typically have a durometer hardness of around 3-70 on the Shore A scale and polyurethanes around 100 on the Shore A scale. The difference between Shore 60 on the D scale versus shore 60 on the A scale is approximately a factor of 300. Qualitatively, this is the difference between a hardhat and a rubber band. Therefore, tethers made from low hardness materials say 150 or lower on the A scale or 1 or lower on the D scale) are not prone to “cheese grate”, erode, cut or remodel tissue. Silicones and polyurethane, therefore, make ideal materials for tether 14 if long-term implantation of device 10 is desired. Tether 14, anchoring element 18 and device body 12 can be made from different materials or different shore hardness of the same material. For example, in FIG. 15a-e tether 14 and anchoring element 18 can be silicone shore hardness A70 and device body 12 can be shore hardness A30 so that the strength of tether 14 and anchoring element 18 are maximized and yet device body 12 is soft enough to easily deform in the GI tract and not cause mucosal erosions or large axial forces on tether 14.

Anchor element 18 can be made from the same material as tether 14, and then the region around anchor element 18 and the connection to tether 14 can be protected with a permanent or temporary external sheet, foil or conformal coating of a thin (e.g. 0.02 to 0.05 mm thick) metal or harder polymer such as PTFE, nylon or polyurethane while in the slotted delivery needle. Such a protective layer prevents the very soft material, e.g. silicone, from being nicked or cut by the potentially sharp sides of the slotted needle during the delivery procedure. Any nick in silicone quickly propagates into a full tear under a tensile load.

In a preferred embodiment, tether 14 is made of a highly elastic and soft material, such as silicone, polyurethane or equivalent, and will extend like a rubber band, thereby minimizing the forces acting to rip device 10 out of its anchored position. Typical diameter of tether 14 can be anywhere from 0.1 mm to 5 mm, preferably 1-2 mm.

Tether 14, backstop element 16, anchor element 18, device body 12 or the connection between any of the former can be non-absorbable and therefore permanent. Alternatively, they can be temporary (absorbable, acid sensitive, fused, etc.) and designed for degradation and eventual automatic or externally triggered release of the device after a set time of implantation.

Various configurations of device body 10 and tether 12 can be provided in the form of a kit. Each kit can include a device body 12 of a specific size and shape as well as surface texture and a tether of a specific length, diameter and elastic/non-elastic characteristics together with a positioning and delivery device.

Tether 14 length and device body 12 size are preferably selected such that when device body 12 resides within the duodenum, it does not migrate distally more than the first 10 cm, preferably, first 5 cm of the duodenum to avoid interfering or irritating the papilla of Vater. If device body 12 or a sinker element (as described below) does extend beyond the first 5 cm of the duodenum, it is preferably positioned far enough beyond the papilla of Vater to not touch it and cause potential inflammation of the area.

It is possible to attach a “sinker element” to device body 12 that continues beyond the first 5 cm of the duodenum to enable the small intestines to apply a force keeping an antrum-anchored device 10 pointed towards the pylorus or running fully or partially through the pylorus in the steady state. A thin silicone tube, say 1 mm in diameter and 5 to 20 cm long with a hollow or solid ball 5 to 20 mm in diameter at its distal end is one example of such a sinker element. Normal peristaltic forces in the small intestines would act to keep the sinker line under tension which would pull device body 12 caudally and bias it to resist the forces of retrograde propulsion. Alternatively, tether 14 can be attached to or extend through portions of the GI tract, downstream of the pylorus all the way to the anus, to bias tether 14 in the caudal direction.

Tether 14 can be anchored at both its proximal and distal ends to the tissue using in-tissue or through-tissue anchoring. This scheme assures that the range of motion of device 10 in the GI tract is limited by the range of motion of tether 14 between its two anchoring points. Such and anchoring scheme can be implemented by a delivery device having two delivery heads arranged in tandem, one for the distal and one for the proximal ends of tether 14. The delivery head for the distal end of tether 14 is positioned at the desired site and the distal end of tether 14 is anchored to the tissue. Then the delivery device is repositioned and the delivery head anchors the proximal end of tether 14 to the tissue in the desired location. The delivery device needs to be intubated into the patient just one time in such a configuration. For example, the distal end of tether 14 can be in the intestines (small or large) and the proximal end in the stomach.

Due to the position of the anchor, the length of tether 14 and the shape and size of device body 12, device 10 or portions thereof will shuttle or move within or between the antrum and duodenum through the pylorus due to natural peristaltic and reflux forces present in the GI tract and thus intermittently contact mucosal tissue of the duodenum, pylorus and antrum.

Such intermittent contacting will be largely influenced by movements of the GI tract (e.g. peristaltic movement of the antrum and duodenum as well as sphincter movement of the pylorus) as well as flow of chyme from the antrum to the stomach body (retropropulsion) or from the antrum to the duodenum and bile reflux from the duodenum to the antrum. Flow of chyme from the antrum to the duodenum will carry device body 12 or portions thereof into the duodenum, while retrograde flow of bile from the duodenum into the antrum may carry device body 12 or portions thereof into the antrum. Such shuttling back and forth can occur 2 to 3 times per minute when the subject is feeding. Without being bound to a theory, the present inventors are of the opinion that such shuttling will activate receptors present in the pylorus, duodenum and/or antrum and thus reduce eating rate and/or eating amount.

In a further embodiment, device 10 comprises device body 12 which resides in the duodenum intermittently contacting mucosal tissue of the duodenum and pylorus and does not shuttle into the antrum. In this configuration of device 10, tether 14 is attached to pylorus, antro-pylorus or duodeno-pylorus tissue (mucosa, submucosa and optionally muscle) and is long enough to allow device body to intermittently contact the wall of the duodenum and the pylorus (at the duodenal side). Device body 12 can be shrunk to dimensions of tether 14 and therefore contact of the GI tissue with tether 14 alone (or tethers if multiple devices are present) will be sufficient to create the desired change in the eating behavior of the subject. Device body 12 can be a gradual enlargement of tether 14, for example a conical tube starting 1 mm in diameter and growing up to 30 mm in diameter, and either open ended at its distal end, solid or sealed and hollow.

It will be appreciated that device 10 can also be configured to enable application of back pressure on the pylorus wall (at the duodenal side) especially when bile flows from the duodenum to the antrum. The duodenum does not normally contain food particles, and therefore any such pressure or mechanical stimulation of a solid substance may generate a signal that solid food has managed to get into the duodenum, and that in turn may drive signaling to slow down gastric motility in general.

In a further embodiment, device 10 comprising device body 12 resides in the antrum intermittently contacting mucosal tissue of the antrum and possibly the pylorus and by virtue of the location of anchoring and the length of tether 14 is not capable of reaching the duodenum. In this configuration, tether 14 is attached to tissue of the antrum and is long enough such that device body can only contact the walls of the antrum, as well as walls of the pylorus (at the antrum side and optionally the opening).

It will be appreciated that the present invention can also utilize configurations which include several implanted devices where each device can be separately positioned in a specific location of the antro-duodenal region. For example, multiple such devices 10 can be positioned around the pyloric opening to collectively obstruct the lumen. In one embodiment, four device 10 implants each with a 10 mm diameter device body 12 will block ⅔ of a 25 mm diameter fully open pylorus.

Another example of a multi-device configuration includes 2-5 device 10 configurations each including device body 12 shaped as a disk or volume occupying element such as a sphere. Each of the devices can be individually tethered to one or more tissue locations in the antrum, pylorus or duodenum. The effective volume and surface area of the combined devices is increased linearly with the number of devices implanted, which increases their stimulation or flow blocking capability, while the size of each individual device 10 is kept to a minimum to enable easy introduction into the stomach, easy anchoring, minimize the forces on each device 10, and ensure that each device 10 is small enough to safely pass through the small intestine if it becomes detached from the tissue to which it is anchored. In one embodiment, one or more disk-shaped device bodies 12 are positioned around the pyloric opening forming a leaf valve (analogous to a tricuspid heart valve for example) for reducing flow through the pylorus.

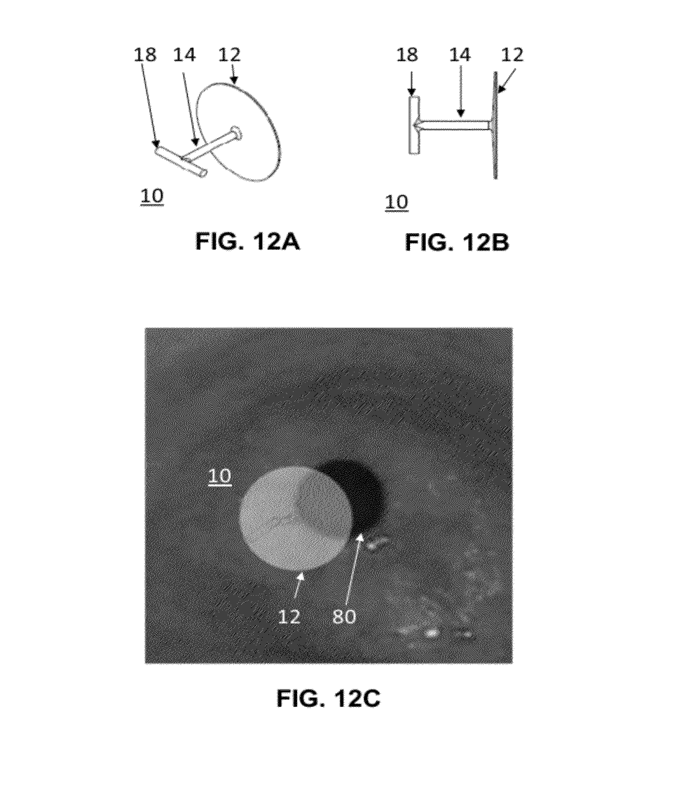

It will be appreciated that although the device configurations described above include a tether 14, it should be noted that direct attachment of device body 12 to the tissue is also contemplated herein. Such direct attachment can be realized using clips, staples, barbs, sutures and the like. For example, the “pyloric disk” referenced in FIGS. 12a-c can be directly attached to the stomach side of the pyloric tissue with one of more barbs, pincers, clips or sutures. Such attachment can prevent rotation of device body 12 which would enable it to be shaped asymmetrically just to the side that causes partial blocking of the pylorus opening, as opposed to the rotationally symmetrical device body 12 depicted in FIGS. 12a-c. By reducing the surface area of device body 12, forces acting on device 10 are therefore substantially reduced which lowers the forces trying to remove device 10 from its anchored position.

Several approaches for implanting device 10 are contemplated herein, including open surgery, laparoscopic surgery and endoscopic surgery.

In the open surgery approach, a physician gains access to the antro-pyloro region through a full incision and anchors/sutures end 16 of tether 14 to the antral or pyloric tissue. Following anchoring the physician places device body 12 within an antral, pyloric or duodenal region and closes the stomach and skin incisions.

The laparoscopic approach is largely similar with the only major difference being replacement of the open incision with three or more small incisions through which the device can be guided into position using laparoscopic equipment.

A combined laparoscopic and endoscopic procedure can be used as well. A laparoscope can be introduced and positioned so that it impinges on the external surface of the stomach where the anchoring is desired. The impingement can be viewed from inside the stomach using an endoscope and the endoscope maneuvered to anchor the device at the point of impingement. The laparoscope can then be used to manipulate the anchor on the external surface of the stomach (for example guide it back into the stomach) or simply to confirm that the anchor site has no long term bleeding or other complications. Alternatively, the laparoscope can be used to introduce tether 14 in through the exterior of the stomach lumen, say in the distal antrum for example, while leaving anchoring element 18 on the outer surface of the stomach, and device body 12 can be attached to tether 14 from inside the stomach using endoscopic means.

Regardless of the approach used for delivering device 10, once device 10 is positioned, tether 14 is attached to the mucosa/submucosa and optionally muscle using sutures, staples, clips or by running anchoring element 18 of tether 14 through the tissue and anchoring it within the tissue or providing or deploying a backstop element 16 that sits on the surface of the tissue.

Any of the above surgical approaches can be effected using two separate procedures. In a first procedure, an anchor for tether 14 is established, while in the following procedure, device 10 is reversibly or permanently attached to the anchor point.

The above described devices can be anchored to stomach tissue using non-elastic or elastic tethers. Preferably, the device of the present invention is anchored to stomach tissue via at least one tether configured for elastic compliance and of a softness to not allow for cutting of the tissue tract by tether 14. Human tissues are dynamic and the forces and strains generated by tissue movement can be large enough to cause hard or non-compliant sutures or tethers to cut through or rip out of tissue or erode surfaces such as mucosa. This is especially true if a non-compliant suture or tether attempts to constrain the normal motion of the tissue or is in a geometry that does not allow for relative motion between itself and the tissue. Therefore, compliant anchoring as taught by the present invention is preferred as it does not constrain the tissue from its natural movement and thus minimizes the chances of anchor failure and tissue erosion.

Anchoring of the device body is effected using an elastic tether 14 which is attached to a tissue anchoring element suitable for providing in or through-tissue anchoring capabilities. Examples include t-bar structures, barbs, coils, pig-tail structures (normally coiled and linearized when forcibly pulled or inserted in a delivery needle), umbrellas, balls or baskets (expandable, static, hollow, solid or wire) screws, augers, or any other structures capable of residing in or against a tissue and opposing a force applied thereto in one or more directions, whether designed to be permanent or removable. The tissue anchoring element can be fabricated from any material including metals, alloys, polymers and the like. The anchoring element structure can be rigid, compliant or elastic in nature. The anchoring element can be constructed from a combination of materials which provide the rigidity necessary for resisting forces applied to the anchor while maintaining a soft non-traumatic interface with the tissue, thereby minimizing tissue abrasion. For example, a t-bar anchoring element can be constructed by overmolding a rigid plastic or metal bar or wire with silicone to form a T which has a silicone covered cross bar and a silicone tether stem. The use of overmolded metal also provides the anchoring element with radio-opacity and thus enables identification thereof using imaging techniques. Alternatively part or all of the anchor, tether and body of the device can include a radio-opaque material during fabrication, such as barium sulfate or a small metal ball or wire element. It is important that the outer surface of the anchoring element make a smooth transition to the stem to avoid having an edge catch on the entry hole in the tissue and increase resistance of the delivery needle puncture. Such resistance can be difficult to overcome by pushing at the remote end of a long pushrod at the proximal end of the endoscope, and large forces can separate anchoring element 18 from tether 14 during needle insertion. Therefore a single molded piece comprising anchoring element 18 and tether 14 is preferred.

The loading capabilities of the anchoring element are determined by a combination of structure, size and choice of materials. It will be appreciated that such loading capabilities can be designed into the anchoring element according to use and site of anchoring.

Device 10 includes a device body 12 which is anchored into tissue of the GI tract. In a preferred configuration of device 10, tether 14 is provided with an anchoring element 18 which enables through-tissue anchoring. To enable through-tissue anchoring, anchoring element 18 and attached tether 14 are delivered from within the stomach through stomach wall tissue or a GI sphincter (as described hereinbelow) and anchor element 18 is deployed and juxtaposed against the outermost tissue layer (serosa) of the stomach, through and against the inner luminal surface of an invaginated tissue fold in the lumen of a GI tract, or against the backside of a sphincter such as the duodenal side of the pyloric sphincter or the stomach side of the lower esophageal sphincter. A washer element may be used to increase the surface area of the contact of anchoring element 18 against the tissue as will be described later.

The washer element can also be made of two parallel surfaces that are temporarily and elastically bent at 90 degrees from each other when mounted in the delivery device (see for example washer 208 in FIG. 16b). Anchoring element penetrates the first washer 208 surface at a normal angle and then after detachment from the delivery device the second surface of washer 208 folds flat and covers anchoring element 18 as a flap that rests parallel to the underlying first surface of washer 208, thus returning to the original relaxed position. In this manner, the anchoring element is sandwiched between the first and second surfaces of the washer and not exposed to direct rubbing against the mucosal surface, which could lead to tissue erosion or disintegration of the anchoring element. Alternatively, a ridge can be raised along the circumference of the washer element so that the anchoring element is recessed lower than the ridge and is also in this manner protected from directly rubbing against the mucosal surface.

A washer may be made of any solid, porous or mesh-like material that effectively increases the surface area of anchoring element 18 against the tissue to prevent anchoring element 18 from being buried in the tissue. Such burrowing into the mucosa or submucosa has been observed by the inventors in pigs for devices anchored with small (e.g. a 1 mm diameter×8 mm long T-anchor) anchoring elements placed directly on the mucosa or submucosa of the stomach. Burrowing of anchoring element 18 makes its endoscopic retrieval and removal from the stomach difficult if not impossible, detracting from the advantage of full-reversibility inherent in through-tissue anchoring where both ends of the tether emerge into the lumen of the organ. Alternatively, for a more permanent in-tissue anchoring, such burrowing of anchoring element 18 into the tissue may be a desirable feature, in which case no washer should be used.

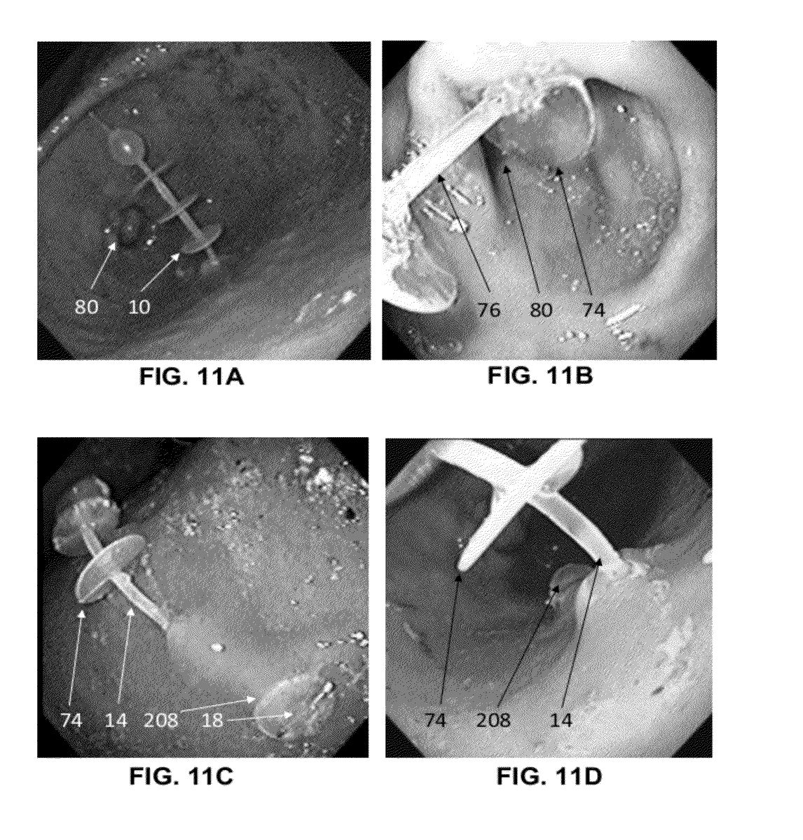

Through-tissue anchoring is preferred for its anchoring strength and full reversibility. While experimenting with several tissue anchoring designs, the present inventors have discovered that devices anchored within stomach tissue (e.g. through stomach wall, a tissue fold or through a sphincter) using through-tissue t-bar anchoring and elastic tethers resulted in consistent anchoring results while minimizing tissue necrosis and damage at the site of tissue penetration (see FIGS. 11c-d for an example of this).

Anchoring through a sphincter or a tissue fold is advantageous in that the anchoring element is maintained on the luminal side of the GI tract. This feature ensures that the anchoring element is released into the GI tract when disconnected from the tether and can be recovered or harmlessly passed out of the body. Keeping the anchoring element on the luminal side of the GI tract can also be achieved with stomach wall anchoring by simply delivering the anchoring element and attached tether out of the stomach through a first hole and back in through a second hole. This can be effected by forming a tissue fold from the stomach wall, through combined laparoscopic/endoscopic means, or by utilizing a device that provides stitch-like functionality. The tether can be anchored to the tissue using an anchoring element or the tether can be secured via knotting or the like.

The tether can also include a stopper structure on the tether for limiting movement of the tether and/or anchoring element in an unwanted direction. This stopper feature is important, particularly as it has been discovered by the present inventor that the omentum tends to pull any objects, such anchor element 18, away from the serosa surface of the stomach. A stopper on the portion of tether 14 that is inside the stomach lumen would prevent further migration of the tether outside the stomach due to the pulling forces of the omentum on anchoring element 18. Additionally or alternatively, a washer can be used for preventing an anchor positioned against a tissue (e.g. a t-bar anchor) from burying into and eroding out of the tissue under the pulling forces of the tether. One configuration of such a washer is shown in FIGS. 3a-c which is further described in the Examples.

In a further embodiment, device 10 consists of up to 1,000 device bodies 12 that are effectively like little spaghetti noodles 1-15 cm long that are tethered at one end close to the pyloric opening. Each device body 12 can have a round, oval, square, rectangular, triangular or irregular cross section and also vary in width, shape and/or thickness along their length. Device bodies 12 can be hollow and open at one end or sealed and filled with a gas, liquid or a gel to increase their volumes and/or compliance along all or part of their length. The advantage of multiple device bodies 12 is that the force on each device body 12 is relatively small and therefore the anchoring can be shallower, perhaps into the submucosa layer only, and hence simpler. Furthermore, should any anchor 18 or tether 14 fail, there will remain sufficient device bodies 12 in the region to perform the function of device 10. Dislodged device bodies 12 will pass harmlessly through the digestive system. Furthermore, device 10 of this configuration will not be blocked by food particles because device bodies 12 will be free to spread apart and rearrange themselves as the pylorus opens and closes and based on the prevailing axial flow of food through the pylorus. Assuming that each device body is an open ended thin walled tube 2 mm in diameter and that the maximum pyloric opening on average is 10 mm in diameter, then a quantity of approximately 25 device bodies 12 would fill the pyloric opening and delay gastric emtpying.

In this specific embodiment, the device bodies 12 are thin walled tubes a few mm in diameter and up to 15 cm long that are sealed on the stomach side and open on the duodenal side and are easily crushable by stomach tissue and the pylorus, and therefore let the latter seal properly. By being soft and crushable, device bodies 12 will not cause erosions. The tubes when not crushed and when running through an open pylorus take up cross sectional area of the lumen and effectively bulk the pyloric opening from the inside of the lumen and thus partially obstruct flow through the pylorus. The tubes can be long enough to have their distal end reside most of the time in the duodenum, thereby assuring that the device bodies 12 run through the pylorus at all times. The tubes forming device bodies 12 can have stacked disks or cones on them to further increase their ability to block flow.

Implantation of device 10 in this embodiment can be effected endoscopically by pushing in all of anchoring elements 18 of device bodies 12 simultaneously into the tissue proximal to the pylorus preassembled in a radial pattern around the pyloric opening or else served up in a “magazine” format inside the stomach and anchored one at a time. In the latter embodiment, 25 device bodies 12, each 2 cm long and 2 mm in diameter, can fit single file end-to-end into a hollow tube, say 2.2 mm inner diameter and 0.5 meters long that fits into the working channel of a standard gastroscope and are presented with the anchor side first to the tip of the working channel. Anchor element 18 is secured into the tissue, the endoscope is then retracted 2 centimeters, device body 12 is pulled out of the tube and remains tethered to stomach tissue by anchor element 18 (e.g. a barb), and the next device body 12 is presented to the tip of the working channel of the endoscope and the cycle repeated until sufficient device bodies 12 have been delivered into the pyloric region. Alternatively, all or a portion of device bodies 12 can be attached to a single anchoring element 18 through one or more tethers 14.

Reference is made to FIGS. 2a-c and 3a-c showing system 100 which comprises positioning and delivery device 50 and device 10. Delivery device 50 is designed to fit on an end of endoscope 68 and to communicate with one or more of its working channels, whether internal or external. Delivery device 50 can be molded, machined or fabricated from a polymer or metal or a combination thereof. Delivery device 50, or portion thereof, can be rigid or alternatively elastic to make insertion into the GI tract easier. The front end of delivery device 50 is preferably blunt and rounded to enable smooth delivery through the GI tract.

Delivery device 50 includes vacuum chamber 52 which is designed for invagination of organ tissue 82 as a tissue fold into vacuum chamber 52 via vacuum applied through vacuum conduit 54 which is attachable to a vacuum hose which runs outside and along the endoscope or within a working channel thereof. Vacuum chamber 52 can optionally contain multiple vacuum conduits 54, channels or ports running along the top or sides of vacuum chamber 52 to allow for uniform distribution of vacuum along the entire length and breadth of vacuum chamber 52. Such channels or multiple ports will not be sealed by the tissue upon it being sucked into the vacuum chamber and therefore allow for the suctioning of a uniform volume of tissue into the vacuum chamber and a stronger counterforce to the penetration of delivery needle 206, which would otherwise easily break the vacuum in vacuum chamber 52 if the vacuum is delivered to just one area of vacuum chamber 52. Alternatively, the top and sides of vacuum chamber 52 can be formed from channels or tubes with a screen or porous mesh material to allow for uniform distribution of the vacuum force along the vacuum chamber volume without having any one point of vacuum entrance sealed by the tissue and therefore block the vacuum reaching other parts of vacuum chamber 52. Tissue is sucked into vacuum chamber 52 in a direction normal to the axis of movement of needle 206.

The anchoring is most stable and least likely to erode when the maximal force on device body 12 acts in the direction from backstop 16 to device body 12 and stretches tether 14 straight. If the force, such as peristalsis or antral contraction waves, acts on device body 12 is in the direction of backstop 16, then tether 14 will fold back on itself and tend to erode the tissue at the 180 degree bend where stresses will be concentrated. Therefore, it is important to analyze the direction of the maximal forces on device 10 prior to selecting an anchoring scheme. System 100 allows anchoring in both of the directions described above (as demonstrated in FIGS. 2 and 3 respectively). Although tether 14 is flexible and allows device body 12 to move in any direction, a first embodiment of system 100 illustrated in FIG. 2a-c has device body 12 tethered in a direction that it is pointing away from the delivery endoscope (caudal direction) when tether 14 is stretched straight through the tissue fold. This anchoring scheme is most useful when the maximum force acting on device 10 is in a direction away from the delivery endoscope. This anchoring scheme is also useful if it is important to locate device body 12 further away in the caudal direction from the anchoring site, for example as close as possible to the pylorus while still maintaining anchoring within the stomach tissue and not in the duodenum.

In this first embodiment shown in FIGS. 2a-c and 5a-c, delivery device 50 further includes a device chamber 56 which is configured for holding device body 12 under frictional forces. Device chamber 56 is domed on the distal end to allow easy passage through the GI tract and also to provide a counterforce to the force of the needle penetrating device body 12. Device chamber 56 and vacuum chamber 52 are configured such that a device body 12 residing within device chamber 56 seals off vacuum chamber 52 and enables it to apply the vacuum force necessary to pull in tissue. Furthermore, vacuum chamber 52 and device chamber 56 are along the same axis to allow the needle to travel in a straight path from the working channel and through vacuum chamber 52, device chamber 56 and device body 12 without applying side forces to device body 12 that may dislodge it from chamber 56.

Delivery device also includes needle guide 58 designed to enable penetration of a delivery needle 206 through a tissue fold contained within first chamber 52 and into device body 12 held within device chamber 56. Delivery needle 206 is slotted such that anchoring element 18 can be inserted thereto with tether 14 being dragged along through the tissue parallel to and outside needle 206.

The force of the vacuum or the distance between the needle penetration axis (height of needle) with respect to the roof of vacuum chamber 52 can be varied such that needle penetration of a tissue fold held within vacuum chamber 52 can be at any preselected tissue depth or through any tissue layer. For example, in anchoring to wall tissue of the stomach, the height of the axis of delivery needle 206 can be adjusted such that tether 14 passes through submucosal tissue, muscular tissue, or clear through to the outside of the serosa, before remerging back into the stomach.

Within delivery needle 206 is a plunger (not shown) to release anchoring element 18 once delivery into device body 12 is effected. A pocket 60 positioned on the side of delivery device 50 is designed for releasably holding backstop element 16.

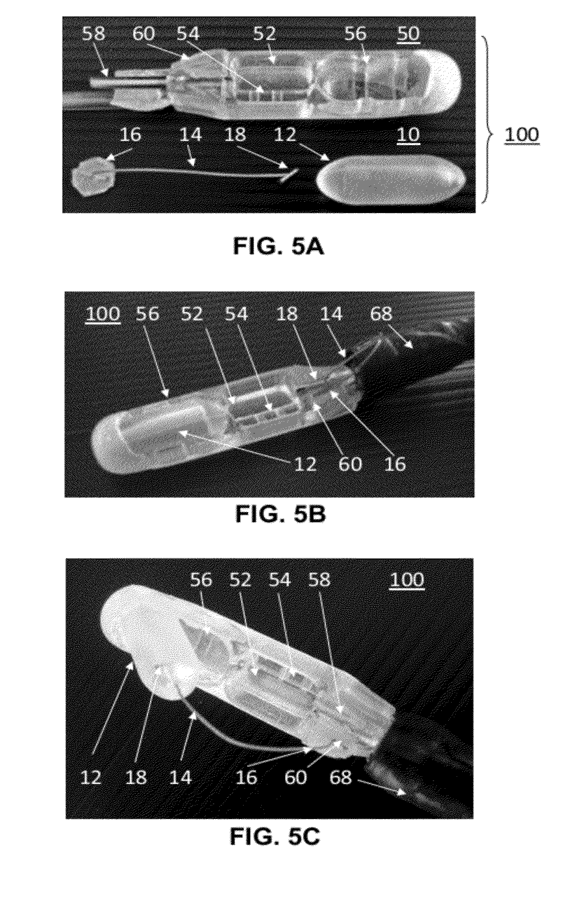

FIG. 5b illustrates device 10 loaded into delivery device 50. Tether 14 and device body 12 are separately attached to delivery device 50, prior to the procedure.

To position and anchor device 10, an endoscope mounted delivery device 50 loaded with tether 14 and device body 12 as shown in FIG. 5b is positioned within the lumen of the stomach and vacuum is applied through a vacuum hose attached to vacuum conduit 54. Once a tissue fold is sucked into vacuum chamber 52, delivery needle 206 carrying anchoring element 18 is penetrated through the tissue fold and into device body 12. A plunger delivered through delivery needle 206 is used to push anchoring element 18 out of delivery needle 206 and into hollow space of device body 12. The vacuum is released and the endoscope is gently pulled back to allow device body 12 to release from second chamber 56 and backstop element 16 to release from pocket 60. FIG. 5c shows device body 12 and backstop element 16 in the process of being partially released from delivery device 50. In this picture, tether 14 would be running through the tissue fold (not shown for clarity). Following release from delivery device 50, backstop element 16 rests against the tissue at the site of needle entry to function as a backstop, thereby effectively anchoring device 10 in the tissue.

Example 1 below describes delivery of device 10 into stomachs of female pigs using an endoscope mounted delivery device 50. Example 2 below describes the delivery of device 10 into the stomach of obese human patients using the same endoscope mounted delivery device 50.

In a second embodiment shown in FIGS. 3a-c, 9a-b and 10a-d, delivery device 50 is designed to deliver a device 10 as pictured in the embodiment of FIGS. 8a-b. Furthermore, the anchoring is such that device body 12 is pointing towards the delivery endoscope (orad direction) as it was discovered by the present inventor in the course of Example 2 that there are strong forces in the stomach in the orad direction during the retropulsive grinding antral contraction waves. This is a non intuitive discovery since conventional wisdom is that the stomach pushes food forward in the caudal direction, yet maximal forces by objects in the antrum are in the orad direction during retropropulsion and grinding of food in a direction away from the pylorus towards the antrum. Therefore, the anchoring direction of having device body 12 pointing in the orad direction was chosen to allow for the maximal force to act on a straight tether 14, and not on a tether 14 bent over on itself by 180 degrees which will concentrate stress and cause erosion at the site of the bend.

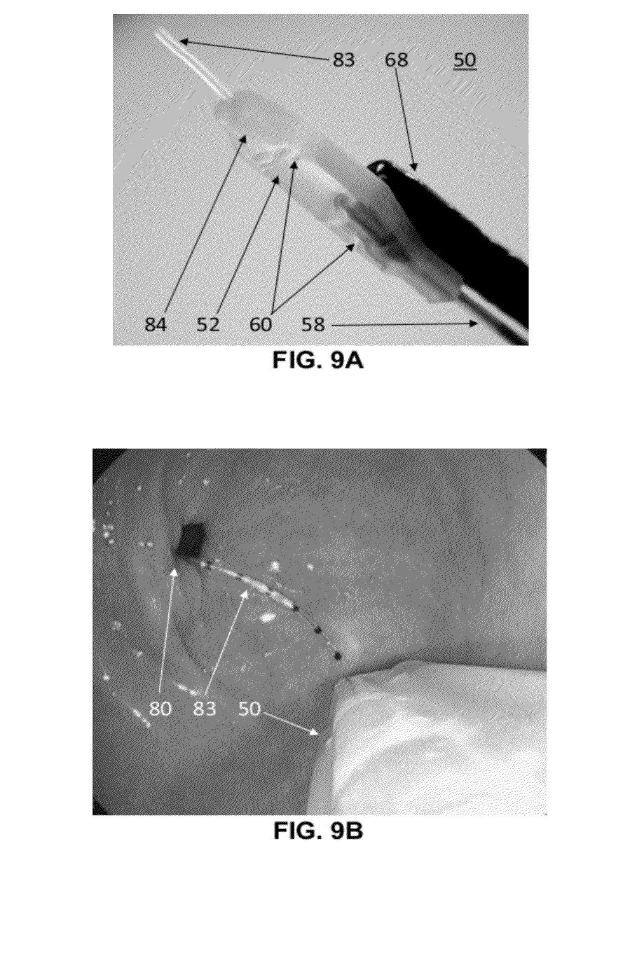

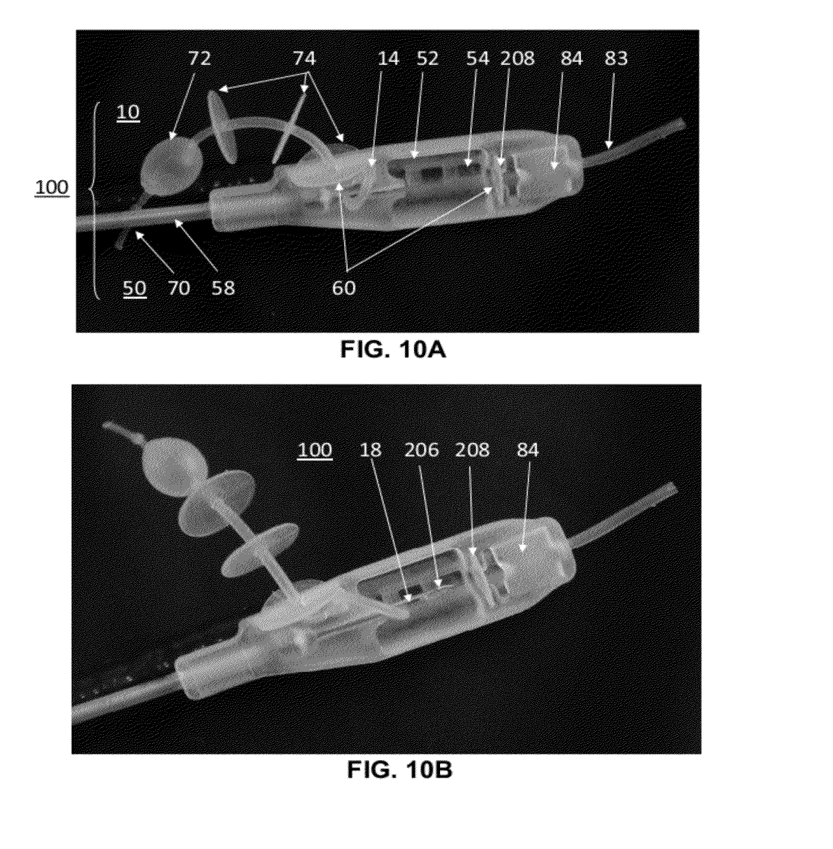

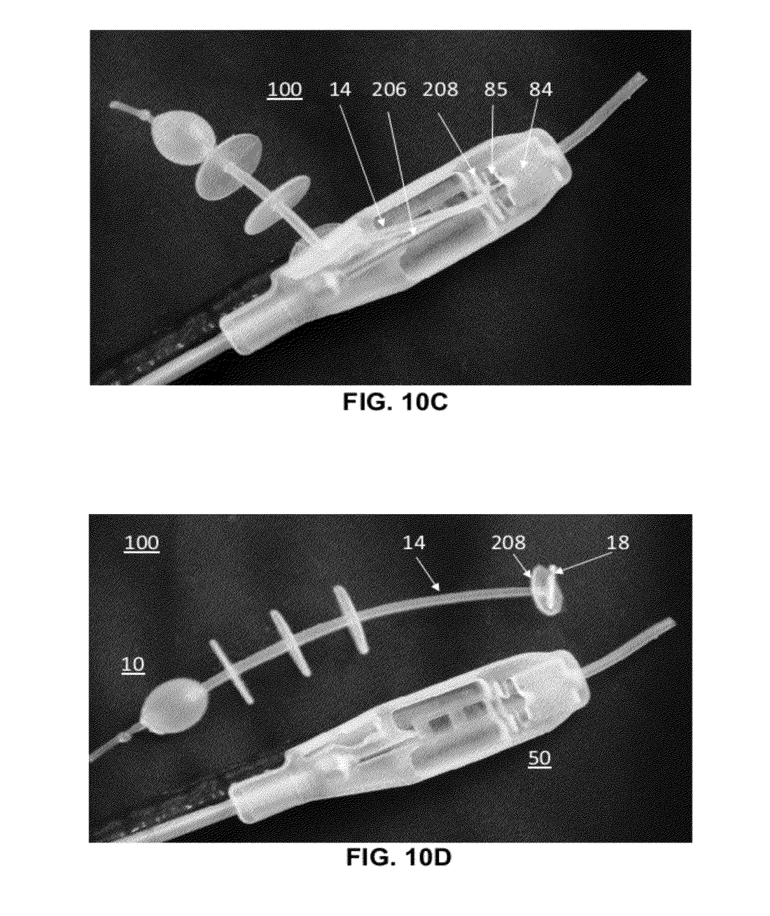

FIGS. 9a and 10a-d show features of this second embodiment of delivery device 50 which includes needle guide 58 that runs externally to the working channel of endoscope 68. Vacuum chamber 52 is connected via a short tube to the working channel of endoscope 68 so that vacuum is controlled by the endoscopist in the conventional manner through the buttons on the top handle of endoscope 68.

Alternatively, vacuum chamber 52 can be connected to a vacuum pump through an external tube that runs outside and parallel to endoscope 68 to leave the working channel of endoscope 68 free for other tools or to enhance the cleaning of the endoscopy lens using the standard water/vacuum controls of an endoscope. In this instance, delivery device 50 fits completely outside the endoscope and is positioned far enough in front of the endoscope to allow for a good field of view for the operator. Delivery device 50 can be attached to the front of endoscope 68 using a flexible attachment that allows for limited angulation of delivery device 50 relative to endoscope 68 to make intubation easier.

In a further embodiment, delivery device 50 can be intubated and its position controlled separately from endoscope 68. In other words, delivery device 50 can have pitch, yaw and roll controls just like an endoscope to enable proper positioning in the GI tract. Position of delivery device 50 can be verified using an endoscope that is intubated beforehand or afterwards and runs in parallel to delivery device 50 and views it from a short distance behind it. Alternatively, delivery device 50 can have visualization means, such as lights and a camera, integrated directly into it for operation as a fully independent device without the need for a separate endoscope.

Delivery device 50 also has pockets 60 for the placement of disk 74 that is part of device body 12 and for washer 208. FIG. 9b shows an operator's endoscopic view of delivery device 50 in a human stomach demonstrating how delivery device 50 does not block the light source and does not block more than 25% of the endoscope's field of view, which makes it very easy to locate the proper point of anchoring in the stomach. Delivery device 50 only extends 3 cm from the tip of endoscope 68 and has maximum cross sectional dimensions of 1.3 cm wide and 1 cm tall, making intubation easy and safe without the use of an overtube. FIG. 9b also shows the endoscopic view of distance marker 83 with a mark every 5 mm that extends beyond the distal tip of the hard portion of delivery device 50 that enables the endoscope operator to properly position delivery device 50 in the stomach relative to a landmark such as pylorus opening 80. Distance marker 83 can be retractable during intubation and then extendable by running it in or alongside the working channel of the endoscope to the proximal end of the endoscope to make it easy for the operator to extend a distance marker to the desired object based on direct visual assessment and to then read the distance from the tip of the cup or endoscope using markings on the distance marker next to the operating handle of the endoscope. Thus when distance marker is even with the tip of delivery device 50, the “0 mm” mark is next to the reading line in the region of the operator's handle of the endoscope. When distance marker is advanced 30 mm and touches the opening of the pylorus, the “30 mm” mark will be next to a reading line in the region of the operator's handle of the endoscope. The distance marker can be a plastic or metal wire running the length of the endoscope and only marked or calibrated at its proximal end, which can also be the handle used to extend and retract it. Due to the two dimensional nature of endoscopic images, it is very hard to gage distances in the stomach without the aid of distance marker 83. Without distance marker 83, it may be necessary for the endoscopist in a separate procedure to introduce an endoscopic ruler device and mark the area of anchoring in the stomach using dye injection or by creating a little bleeding wound with a biopsy forceps.

Delivery device 50 further comprises a handle that with one user motion pushes needle 206 forward to the end of its travel, and in a continuation of that same user motion subsequently pushes the internal pushrod that displaces anchor element 18 out of needle 206. A return spring in the handle assembly assures that when the handle is released, the needle is fully and automatically retracted so that there is no chance of damaging the tissue due to an endoscope displacement while needle 206 is still engaged in the tissue.

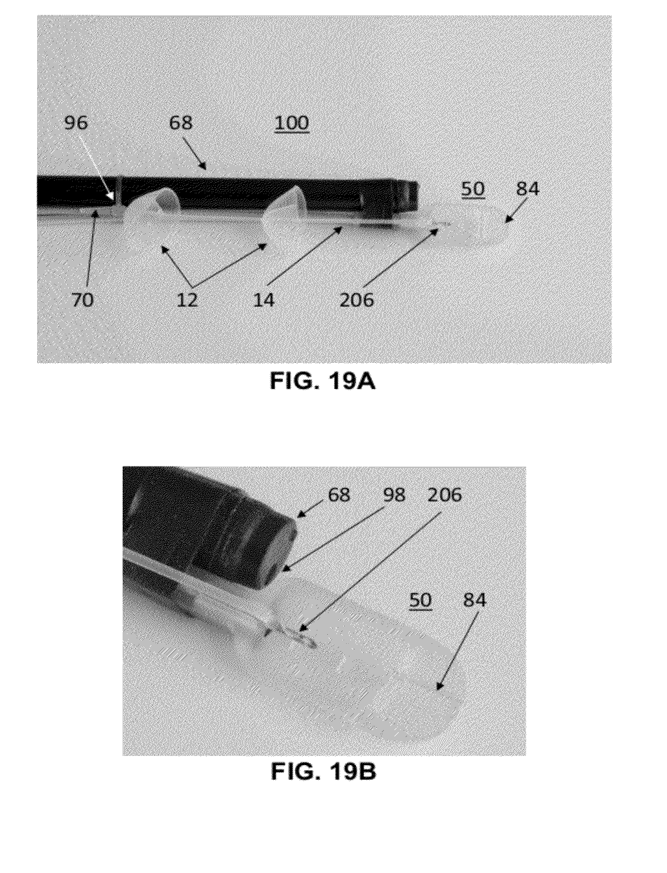

Delivery device 50 further comprises an anchor release chamber 84 where needle 206 enters and anchor element 18 is pushed out of needle 206 behind washer 208. Hollow anchor release chamber 84 ensures that the needle is inside the GI lumen during anchor element 18 release. Without anchor release chamber 84, needle 206 at the end of its travel could be in the tissue or even outside the tissue leading to the release of anchor element 18 in an unintended location. Therefore anchor release chamber 84 provides a defined and protected space within the GI lumen where anchor element 18 emerges from needle 206, rotates to lock behind washer 208 and then is released from delivery device 50 though slot 85 in the proximal portion of anchor release chamber 84. Anchor release chamber 84 can also be a recessed open-faced grove which due to its width of 2 mm or less and depth of several mm does not allow for the invagination of tissue into the area where anchor element 18 is released from needle 206 as is illustrated in device 50 of FIGS. 19 a-b. Open faced groove of release chamber 84 can also be covered with a flexible and detachable covering to prevent tissue invagination into release chamber 84, while at the same time not preventing anchor element 18 from deploying and optionally ripping the covering off release chamber 84 in the process.

Reference is made to FIGS. 10a-d for a more detailed description of the loading of device 10 on delivery device 50. In FIG. 10a, the rear-most disk 74 of device 10 is secured in pocket 60 of delivery device 50. Front tether 70 is temporarily attached to endoscope 68 or needle guide 58 to keep device 10 pointing backwards in the orad direction upon intubation of a patient with system 100. Washer 208 is introduced into pocket 60 of delivery device 50 as well. All three of these connections (disk 74, front tether 70 and washer 208) to delivery device 50 can be friction fits or mechanical fuses that are strong enough to not be displaced prior to the anchoring procedure, but weak enough to detach once endoscope 68 is pulled away from the tissue after the anchoring procedure since tether 14, which is now anchored in the tissue, provides a counter force to remove device 10 from delivery device 50. One of more features (e.g. pockets, clips, grooves, latches, magnetic clasps, mechanical fuses, Velcro™, etc) of delivery device 50 or on endoscope 68 can act cooperatively to keep device 10 securely in place prior to implantation, but yet still detachable after implantation. The detachment force of any feature of device 10 from the delivery device 50 should be high in the direction along the endoscope axis to avoid inadvertent detachment during intubation or removal of the device in an aborted implantation procedure, but relatively low in the direction normal to the endoscope axis after the implantation procedure has been performed. Detachment force of each detained element in the normal direction should not exceed 10 Newtons, preferably 3 Newtons or more preferably 1 Newton to make the detachment of device 10 easy and safe without excessive pull on tether 14 or the tissue into which it is anchored, or without any further intervention or additional action such as the activation of a release mechanism required by the endoscopist other than the removal of endoscope 68 from the site of implantation. In case endoscope 68 is removed from the GI tract without an implantation procedure being performed for any reason, device 10 is designed to stay affixed to delivery device 50 during the removal of endoscope 68 as well so that device 10 is detached from delivery device 50 in the GI lumen without being properly anchored first. Alternatively, a release mechanism can be operated from the proximal handle of endoscope 68 to release device 10 from delivery device 50.

With reference to FIG. 10b, anchoring element 18 is a t-anchor pushed into a slot of an 18 gauge delivery needle 206 which is seen pushed partially through vacuum chamber 52 but before reaching washer 208 or anchor release chamber 84.

FIG. 10c illustrates delivery needle 206 in its furthest most travel having penetrated washer 208 and entered anchor release chamber 84. Tether 14 runs parallel to delivery needle 206 and through the tissue fold sucked into vacuum chamber 52 (tissue not shown for clarity). Anchor element 18 is pushed out of delivery needle 206 in anchor release chamber 84. Delivery needle is retracted back into needle guide 58 and the vacuum is turned off at this point. When the tissue fold detaches from vacuum chamber 52, it takes tether 14 with it which pulls out anchor element 18 through slot 85 in anchor release chamber 84, and disk 74 and washer 208 out of pockets 60.

FIG. 10d shows device 10 when detached from delivery device 50 with washer 208 in place abutted next to anchoring element 18 in the proper alignment relative to delivery device 50 right after an anchoring procedure. Note that device 10 is pointing towards endoscope 68 (orad direction) which means that any peristaltic or contraction waves in the stomach in the orad direction (retrograde) act to stretch tether 14 in a straight line and seat washer 208 firmly against the tissue. Peristaltic or contraction waves in the stomach in the caudal direction (antegrade) acting on device body 12 fold tether 14 back on itself.

Example 3 below describes delivery of device 10 into stomachs of female pigs using an endoscope mounted delivery device 50 according to this second embodiment.

In a further embodiment illustrated in FIGS. 12a-c, device body 12 is a flat disk (or alternatively a bowl or spherical-shaped element) approximately 15 mm diameter and tapering from 0.5 mm thick at its center to 0.2 mm thick at its circumference. The disk is connected to tether 14 which is approximately 10 mm long and 1 mm in diameter which in turn is connected to anchoring element 18 which is 8 mm long and 0.9 mm in diameter. FIG. 12a shows device 10 in an isometric view and FIG. 12b shows device 10 in a side view. FIG. 12c illustrated the intended position of device 10 so that device body 12 partially blocks pyloric opening 80. Tether 14 runs through the pylorus muscle and anchor element 18 rests on the duodenal side of the pyloric sphincter either directly on the tissue or on top of a washer (not shown) as previously described. Device 10 can be made completely of silicone, polyurethane or other soft polymer and therefore not cause erosion as the pylorus opens and closes and the pyloric channel deforms during peristalsis and food grinding. The extent of blocking of pyloric opening is a function of the diameter of the pylorus in the food emptying phase of digestion, the diameter of the disk forming device body 12, and the distance of tether 14 from the outer edge of pyloric opening 80. The latter two parameters are controllable through proper size selection of device body 12 and the depth of the anchoring cup or anchoring chamber in delivery device 50.

FIG. 13a-c illustrate an additional embodiment of delivery device 50 configured for the delivery of device 10 to the pyloric sphincter. In FIG. 13a, delivery device 50 is mounted on endoscope 68 and brought to within a few centimeters of the pylorus where the distance to the pylorus is gauged using direct visualization or using distance marker 83 (not shown).

As illustrated in FIG. 13b, delivery device 50 is then pushed out of the working channel of endoscope 68 using pusher element 87 that runs and is manipulated either outside or through the working channel of endoscope 68 by the endoscope operator from outside the patient's body. Pusher element 87 can translate delivery device 50 in or out axially and/or rotate it relative to endoscope 68. Delivery device 50 can be turned in such an orientation to minimize overall lateral extension from the endoscope dimensions to minimize resistance to intubation, or turned to minimize obstruction of the field of view of the endoscope operator, and then when close to being in position rotated again to orient delivery device 50 relative to the target tissue of interest. Using the standard endoscope controls of up and down and side to side movement, in conjunction with translation or rotation of pusher element 87, delivery device 50 can be positioned so that the anchoring chamber straddles the pylorus ridge using direct visual guidance by the endoscope operator. The translation of delivery device 50 several centimeters away from endoscope 68 allows for the endoscope operator to have within his/her field of view both delivery device 50 and the antrum/pylorus. Needle guide 58 can run external but parallel to endoscope 68 to a needle operating handle outside the patient's body. Delivery device 50 does not need a vacuum source or vacuum chamber in this embodiment since the pyloric ridge is so well defined anatomically that it is easy to straddle using direct visual guidance. The distance between tether 14 and the edge of pyloric opening 80 is controlled by the distance between the top of the anchoring chamber and the axis of delivery needle 206. Delivery device 50 in this embodiment can have a thin profile, say a few mm thickness when viewed head on in order to properly straddle the pyloric ridge. As illustrated in FIG. 13b, once positioned, delivery needle 206 penetrates the pyloric tissue and carries anchoring element 18 and tether 14 (not shown) to the far side of the pyloric ridge. Upon intubation and exit, delivery device 50 is retracted to be close to endoscope 68 to minimize the length of the device being pushed into or pulled out of the patient.

FIG. 13c illustrates in cut away view the cross section of device 10 as per the embodiment illustrated in FIG. 12a-c located in proximity to the pylorus with device body 12 partially blocking pyloric opening 80 and tether 14 running though pyloric tissue with anchoring element 18 positioned on the duodenal side of the pylorus. As food tries to empty the stomach, device body 12 is pushed against the stomach-side of pyloric opening 80 and partially blocks the opening in the form of a flap valve, leading to delayed gastric emptying and early satiety.

In an alternative embodiment, device body 12 can be positioned on the duodenal side of pyloric opening 80 and food emptying would place tether 14 under tension. Device body 12 in this way will block bile reflux from the duodenum to the stomach.

In a further embodiment, a second device body 12 can be positioned between anchoring element 18 and the duodenal side of the pyloric tissue, thereby leading to a partial blockage of pyloric opening on both the stomach and duodenal sides, which would delay gastric emptying and also block bile reflux from the duodenum to the stomach.

In an additional embodiment based on FIG. 12a-c, device body 12 also has one or more flat elements that extend 10 to 30 mm normal to the flat disk. In this manner, if the pylorus is fully open and the walls of the pylorus are erased (i.e. the pylorus becomes indistinguishable from the lumen leading to the duodenum), then the flat disk will lie flat on the surface of the stomach lumen and the normal element will now extend into the lumen and partially block the flow. Additional elements can emerge from the disk at angles other than 90 degrees to enhance this effect.

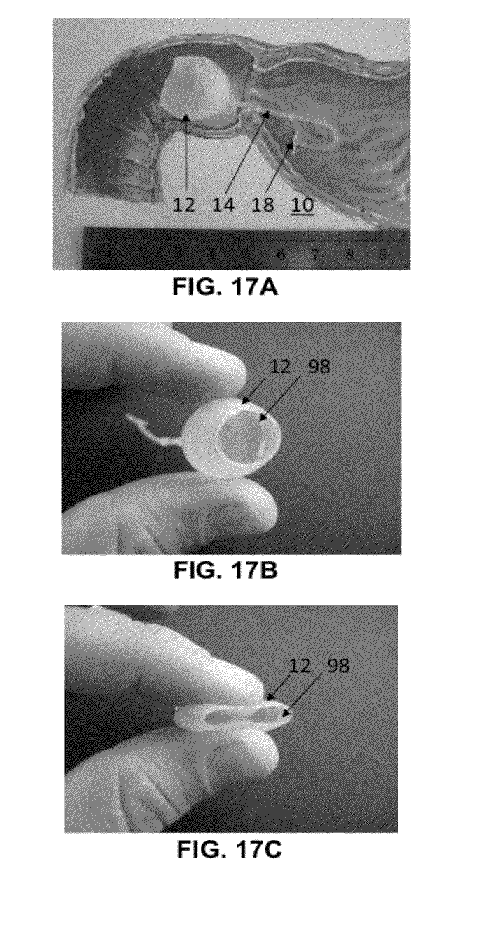

In a further embodiment, device body 12 can take the form of a bowl-shaped element that can effectively block the flow of chyme when either normal or parallel to the lumen. In a yet further embodiment, device body 12 can take the form of a spherical element 10-30 mm in diameter that closely mimics the effect of a gastric polyp that is adjacent to the pylorus. Such a spherical element can be solid, hollow and sealed or hollow and open (for example like the open ended hollow sphere embodiment of device body 12 illustrated in FIGS. 17a-c). When in the open spherical configuration, device body 12 occupies volume in the pyloric canal, but is also very crushable so that it does not present resistance to the peristaltic waves trying to force it into the duodenum. The peristaltic waves act more to crush device body 12 rather than to push it forward.

In yet a further embodiment, device body 12 can be stuck into position without tether 14 adjacent to pyloric opening 80 using a simple barb, clip or suture element that is delivered directly through the working channel of the endoscope for in-tissue anchoring as discussed previously.

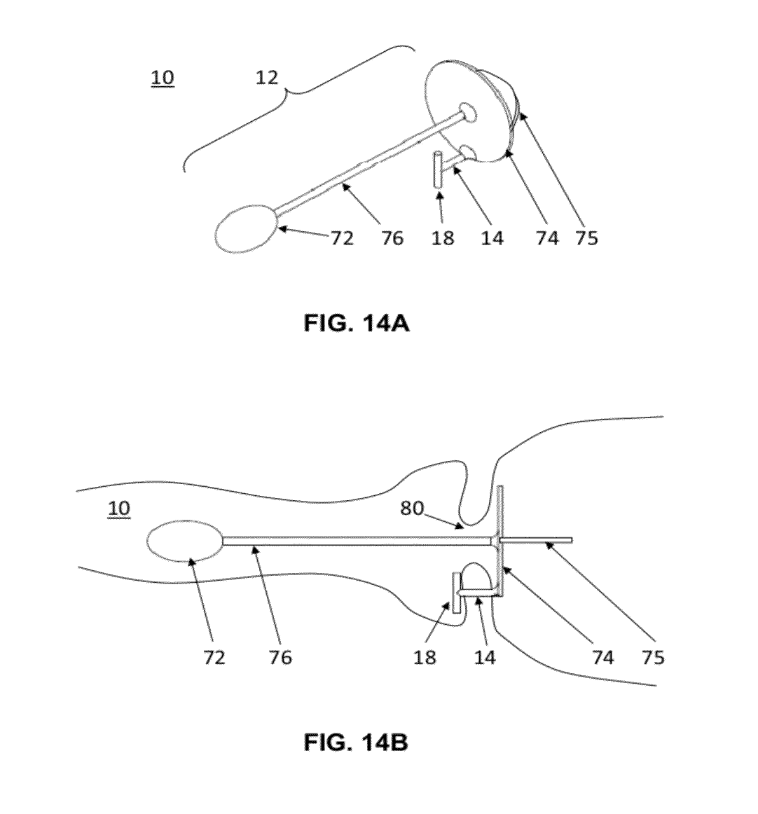

In a further embodiment illustrated in FIGS. 14a-b, device body 12 comprises a flat disk 74 approximately 16 mm diameter and 0.5 mm thick which is connected at its center to stem 76 and front ball 72 and at its circumference to tether 14 and anchoring element 18. FIG. 14a shows device 10 in an isometric view and FIG. 14b shows device 10 in a side view superimposed on the relevant anatomy illustrating the intended position of device 10 so that disk 74 or element 75 partially blocks pyloric opening 80. Tether 14 runs through the pylorus muscle and anchor element 18 rests on the duodenal side of the pyloric sphincter either directly on the tissue or on top of a washer (not shown) as previously described. Stem 76 and front ball 72 are normally in the duodenum (left side of pyloric opening 80) and disk 74 is on the stomach side of the pylorus (right side of pyloric opening 80). Stem 76 and front ball 72 act to align and bias disk 74 against pyloric opening 80, especially during the gastric emptying phase where device 10 acts as a flap valve partially blocking emptying of stomach contents into the duodenum. Front ball 72 can sized to be large enough or be replaced with a forward flat disk similar to disk 74 that will act to retain some of the chyme in the duodenum between the two disks beyond the normal dwell time. This prolonged presence of chyme in the duodenum acts to send satiety feedback signals to the patient and/or slow down gastric emptying as previously discussed. Stem 76 and front ball 72 can be refluxed into the stomach occasionally, but will eventually be transported back into the duodenum due to normal peristalsis or antegrade contraction waves of the stomach. Device 10 can be made completely of silicone, polyurethane or other soft polymer and therefore not cause erosion as the pylorus opens and closes and the pyloric channel deforms during peristalsis and food grinding. The extent of blocking of pyloric opening is a function of the diameter of the pylorus in the food emptying phase of digestion, the diameter of the disk 74, and the distance of tether 14 from the outer edge of pyloric opening 80. The latter two parameters are controllable through proper size selection of disk 74 and the depth of the anchoring cup or anchoring chamber in delivery device 50.

Similar to one of the embodiments of FIGS. 12a-c, disk 74 can have an additional member 75 protruding at an angle relative to disk 74 so that in the event that disk 74 lies flat in the stomach lumen when the pylorus is erased during certain phases of gastric emptying, then one or more elements 75 will protrude into the lumen and partially obstruct the flow. Additional embodiments of device body 12 can be envisioned consistent with the embodiments of device body 12 in the example described in FIGS. 12 a-c.

In a further embodiment illustrated in FIG. 15a-e, device 10 comprises one or more device body 12, which in this embodiment is shaped as a “jelly-fish”, “parachute” or “umbrella” with stiffening webs 94. Device body 12 is highly collapsible and can be made in its entirety, by way of example, from silicone 0.2 to 2 mm thick. Overmolded elements such as the rim of device body 12 or webs 94 can be made or thicker or harder polymers or elastic metals such as Nitinol. The shape and materials of device body 12 enable easy collapse of device body 12 to a cross sectional area of less than 1 cm squared, whereas in the open state illustrated in FIG. 15a, the cross sectional area of 1.5-10 cm squared, preferably approximately 4-8 cm squared. FIG. 15b shows the other portions of device 10, including location of anchoring element 18 attachment, tether 14 and backstop element 16. FIG. 15c illustrates delivery device 50 assembled on endoscope 68 overlaid on a picture of a human stomach properly positioned slightly proximal to pyloric opening 80 with tissue 82 about to be suctioned into vacuum chamber 52. FIG. 15d shows device body 12 in duodenal bulb 90 during antegrade peristalsis where device body 12 acts as a scoop or brake or retention element to capture chyme and extends along tether 14 into the more distal duodenum 92 with each peristaltic wave. When the wave passes, device body 12 returns to its resting state in the small intestines and in the process returns some of the captured chyme with it. FIG. 15e illustrates the position of device body 12 when it shuttles back through the pylorus into the antrum. In this position, device body 12 acts to at least partially block retrograde flow through the narrowing antrum, thereby also interfering with mixing, churning and eventual emptying of food into the duodenum as chyme.

In a further embodiment illustrated in FIG. 16a-c, device 10 consists of a plurality of device bodies 12. In FIG. 16a, each device body 12 is made of 0.3 mm thick silicones shore A50 in the form of a hollow cones with a diameter of 18 mm strung along a common stem 16 and a common tether 14. Each device body 12 acts independently to block the flow of chyme and capture and return chyme proximally due to the elastic nature of tether 14 and stem 16. The location of anchoring of device 10 is illustrated by the position of anchoring element 18 and washer 208. Device bodies 12 can be of the proper quantity and positioned such that when tether 14 is at its resting length, they are only in the small intestines, duodenum 92, the duodenal bulb 90, the pyloric opening 80 or the antrum, or any desired combination thereof. FIG. 16b illustrated device 10 with two device bodies 12 with web 94 that prevents device body 12 from folding back on itself as a result of drag forces. Web 94 only needs to work in tension as the 20 mm OD normally open circumference of device body 12 is maintained by the elasticity of the circumferential rim which in this case is a bead of silicone 0.7 mm diameter, which can resist a force of around 0.01-0.50 Newtons, preferably 0.02-0.2 Newtons and more preferably 0.03-0.1 Newtons before being crushed into an elongated ellipse. Web 94 can be a string-like element and not a solid wall-like element as well. If two web 94 elements are used spaced 180 degrees from one another (e.g. FIG. 16b), device body 12 can still easily collapse to be flat against web 94. If more than two web 94 elements are used (e.g. FIG. 15a), they should be flexible (e.g. very thin or string elements) so as to not hinder the collapse of device body 12. As opposed to a foam element whose resistance to collapse increases significantly with decreasing size, a non porous device body 12 can achieve nearly zero contained volume with minimal force and also does not retain chyme long term (which can lead to bacterial or fungus breeding surfaces) nor do fibers attach to it which can lead to the accumulation of a bezoar. FIG. 16c shows device 10 from FIG. 16b positioned in the stomach where one device body 12 is in the duodenum and the other is in the pyloric antrum.

The function of device 10 can be understood using the following calculation (although based on a dog's GI system, it is assumed to be similar to a human). The small intestine can generate approximately 20-50 inches of water (0.5-1.25 Newtons/cm²) of peristaltic pressure in order to propagate a 2-3 ml bolus up to 12 cm/sec through the small intestines.

When non-caloric non-viscous liquids are used in video fluoroscopy dog studies, the antrum, pyloric and duodenum are relaxed and emptying is rapid. In humans, this implies a minimal pyloric opening of around 2.5 cm diameter and a flow controlled by the pressure exerted due to the gastric tone of the stomach body and antral non-occlusive waves. The duodenum is flaccid and relaxed and so does not resist flow. When a viscous caloric meal is consumed, however, the duodenum, pylorus and pyloric antrum have more tone and are substantially narrowed. Therefore a 2 cm diameter blocking element will effectively interfere with the emptying of caloric contents of the stomach.

However, given that the anatomy is very dynamic, and that the bottleneck and control points that govern the outflow of chyme into and through the small intestines move from being the antrum, the pylorus and the duodenum in a dynamic manner, and that the distance between these elements is not constant during the course of an antrum emptying cycle, it may be beneficial to deploy more than one device body 12 on an elastic tether 14 to always be in vicinity of a bottle-neck region governing gastric emptying.

There are two major kinds of forces the device is exposed to: gastric emptying antegrade bulk-flow propulsion of chyme due to pressures of around 0.015 Newtons/cm² and peristaltic forces of up to 1.25 Newtons/cm². Regarding the antegrade propulsion of chyme, assuming that two 2 cm diameter device bodies 12 in the form of an “open parachute” (12 cm² total open surface area), the bulk flow emptying of the stomach at a pressure of 0.015 Newtons/cm² would generate a maximal force of 0.18 Newtons or 18 grams force, well below the 600 gram breaking force of a 1 mm diameter elastic silicone tether, as determined experimentally.

Regarding peristalsis forces, the same elements must be able to withstand the maximal peristaltic pressures of 1.25 Newtons/cm². Assuming that same two 2 cm diameter device bodies 12 in the form of an “open parachute” now collapse into a “closed parachute” configuration with a total cross sectional area of 2 cm² when exposed to the radial pressure exerted by peristaltic forces, that translates to a linear force of 2.5 Newtons or 250 grams force, also well below the maximal force of 600 grams tolerated by tether 14 of device 10. If device bodies 12 were not collapsible, the peristaltic force would be 7.5 Newtons or 750 grams, above the breaking strength of tether 14, or at the very least increase the probability of tissue remodeling around tether 14 and its eventual explantation from the tissue.

In the embodiment of FIG. 16a-c, the entire device 10 when the cones making up device body 12 compress flat has a volume of between 0.2-4 cubic centimeters, preferably between 1-2 cubic centimeters and is introduced into the body as a functional unit in this minimal volume shape without the need for volume changes (except for elastic folding in pocket 60 of delivery device 50), inflation, swelling, or other means of activation except as provided by the natural elasticity and geometry of the material or materials making up device 10. During routine presence in the GI tract, device 10 can be elastically crushed to this minimal contained volume (down to its displaced volume) without exerting any significant resistive force and return to its original shape once the crushing or grinding force has passed. In this sense, device 10 does not occupy any significant volume in the GI tract, but rather only acts to impede the flow of chyme and/or the grinding of the food in the duodenum and stomach respectively.

As a general consideration for the design of device 10 as discovered by experimentation by the inventor, a balance must be achieved between having device 10 and particularly device body 12 small and soft enough (volume, surface area and collapsibility) to minimize peristaltic drag and resultant forces on the tether and anchor and yet large enough to achieve proper physiological effect. For example, a properly configured parachute-shaped device body 12 is designed to have a large effect on retarding fluid flow in one direction, yet the muscle contraction during peristalsis can easily collapse such a device body itself. Bulk flow in the antegrade direction actually acts to “inflate” a parachute-shaped device body 12, thereby keeping it open and maximizing its effect in retarding the flow. Therefore with almost no structural rigidity required, the effective volume of a parachute-shaped device body 12 when open against flow is large (e.g. a hemispherical parachute of 20 mm diameter has a contained volume of 2,000 cubic millimeters) when it is resisting bulk flow in an open lumen, yet the effective or displaced volume that peristalsis can act against is very minimal when device body 12 folds flat into a semicircular area of 600 square millimeters. On the other hand, it is easier for the stomach muscles to grab a spherical shape 20 mm in diameter and apply peristaltic forces that tend to remove device 10 from its anchoring position.

The notion of resisting gastric emptying by a device body 12 comprising a very flimsy parachute-type element is not intuitively obvious, and rests on the observation that gastric emptying occurs as bulk flow mainly due to the slight pressure differential between the stomach body and the duodenum during the emptying phase, whereas peristaltic-driven events such as antral grinding, or lumen-obliterating contractions during phase III of the MMC or vomiting (which are the highest forces device 10 is subject to in the stomach) act on a minimal collapsed volume of device 10. These two modes of operation (elastic reversion between expanded shape to block emptying and collapsed shape to minimize peristaltic forces acting on device body 12) occur sequentially in a cyclical pattern, and therefore the natural elasticity or ability of the device body 12 elements to switch between open and closed configurations is an important part of the design of device 10.

Another parameter in the proper design of device 10 is to ensure that the force required to displace device body 12 over 3-4 cm against the counterforce of the elastic tether is less than the force required to deform or invert device body 12 beyond its intended range of deformation. For example, if device body 12 is a caudal pointing hollow cone, the force required to invert the cone so that it is orad-facing should be greater than the force to displace the cone 3-4 cm in a peristaltic wave or pressure induced flow. Web 94 in FIG. 16b provides such resistance to inversion of device body 12. In this manner, device body 12 will move distally with the chyme during flow and act as a scoop or retention element to store the chyme in its dead volume with the energy required for such a displacement being stored in the elastic tether, and then device body 12 will return the chyme proximally when the peristaltic wave passes or the pressure-induced flow ceases and the elastic tether returns to its relaxed position. By designing device body 12 so that it is just strong enough to resist deformation at these forces, it can be collapsible enough to not be grabbed by peristalsis traction or to cause any form of tissue erosion in the lumen when it is being crushed by the tissue during peristalsis. Alternatively, device body 12 can itself be elastic to absorb the energy of chyme flow and return some the chyme in the orad direction. Furthermore, when crushed by peristalsis, device body 12 collapses to effectively zero volume and purges any trapped chyme from within the contained volume defined by an open device body 12 and so effectively cleans itself and prevent long term deposition of chyme or other matter in device body 12.

In a further embodiment illustrated in FIG. 17a-c, device 10 consists of an open (hollow) spherical device body 12. The diameter of device body 12, the size of its front opening 98, its wall thickness, the length and diameter of tether 14 and the location of anchoring element 18 in the stomach have all been experimentally determined in order to fit a tight list of experimentally-discovered constraints which apply, in whole or in part to all device 10 configurations described herein:
1. Device body 12 should be open and fully collapsible to a volume of less than 1 cubic centimeter under a radial force of 1N or less to enable easy endoscopic delivery in the collapsed form, to allow for low resistance to the grinding forces of the antrum in order to prevent tissue erosion and overextension of device body 12 away from anchoring element 18, and to allow for purging of the gastric or duodenal contents of device body 12 when crushed by peristaltic forces through front opening 98.
2. Device body 12 should have a large enough front opening (e.g. 1 cm diameter or greater) to scoop up chyme and deliver gastric contents into the duodenum and vice versa when device body 12 shuttles between the duodenum and antrum to stimulate chemoreceptors in these regions. The opening size above is also not prone to clogging by food particles. Furthermore, an open device body 12 is likely to capture sufficient air inside itself to make device 10 float and remain in proximity to the pyloric opening, thereby more effectively blocking it and being in a position to shuttle in and out of the duodenum.
3. As a result of material selection and geometry, device body 12 should have sufficient size and outward springiness (e.g. 0.03N or greater force trying to restore device body from a crushed to a spherical shape, 0.5-1.5 mm thickness walls made from shore A 60 silicone, 10-40 mm, preferably 20-25 mm outer diameter hollow sphere) to assume its natural spherical shape state during the low-pressure bulk-flow gastric emptying events and act as a 0.5-32 cubic centimeters, preferably 2-6 cubic centimeters volume occupying element to partially block the pyloric antrum, the pyloric opening from either the gastric or duodenal side, the duodenal bulb or the proximal duodenum. The blocking can be by physically taking up a cross sectional fraction of the GI lumen, or by gentle pressure exerted by elastic tether 14 on device body 12 which is then transferred to the tissue of interest, for example the duodenal side of the pyloric opening 80. Chyme traversing device body 12 must first displace it against the tension of tether 14, thus increasing the resistance to gastric emptying. The pressures in the GI tract are low enough during gastric emptying that the forces on device body 12 are not sufficient to collapse it (crush force is between 0.1-10N, preferably 0.5-2 N). At the same time, device body 12 at the sizes indicated above is small enough to fit through the pyloric opening (either in its relaxed state or in its deformed or crushed state) and be eliminated harmlessly through the GI tract during a phase III migrating motor complex peristaltic wave if detached from the tissue it is anchored to. Furthermore, device body 12 at the shape and size specified above occupies sufficient volume to be treated as a solid object in the antrum and trigger the lag in gastric emptying that has been experimentally observed when solid objects are present in the antrum, thereby delaying gastric emptying and causing early satiety.
4. Device 10 should preferably have no sharp edges or porous surfaces (e.g. the transition between device body 12 and tether 14 should be gradual and smooth) to prevent fibers from accumulating on device 10 and forming a bezoar over time.
5. Tether 14 should be between 1-10 cm in length, preferably 3-6 cm in length and between 0.5-2 mm in diameter, preferably 1-1.5 mm in diameter and made from an elastic material (e.g. silicone or polyurethane), to enable stretching and a strain of up to 400% as a result of an applied axial force of 1-20N, preferably 2-10N as these forces are routinely encountered in the stomach, especially in the antral regions.
6. Anchoring position in the stomach should be between 0.5-10 cm from the pyloric opening to ensure that device body 10 is present in the pyloric channel and can reversibly and elastically shuttle though the pyloric opening between the duodenum and distal antrum based on the prevailing forces.

Device 10, therefore, is a result of significant experimentation by the inventor as it relates to the processes surrounding gastric emptying, GI anatomy, interaction of the GI system with foreign bodies, and mechanical design. All of the above limitations have led to narrow design constraints that were it not for in-vivo experimentation would be unlikely to be discovered empirically.

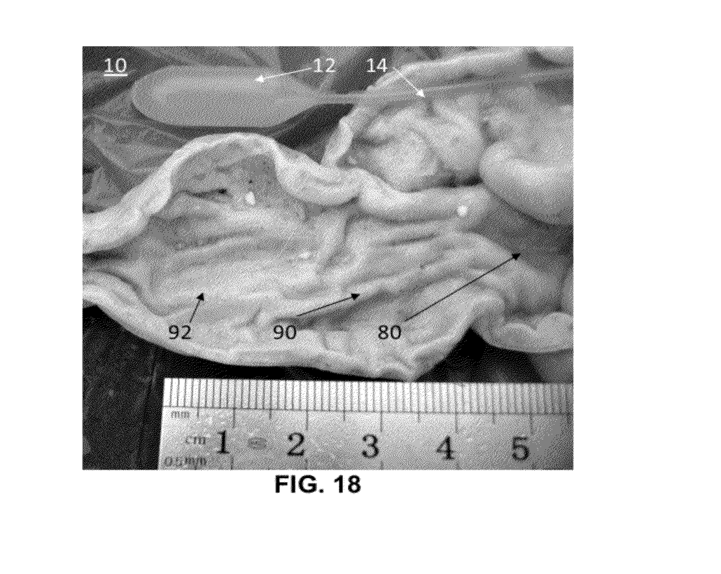

In contrast to the above, prior art “flow reduction elements” (e.g. those described in WO 2006060049) are designed to be stationary relative to the GI tract, which means flow of chyme is impeded through direct flow resistance (obstruction) and/or friction (energy dissipation). Any flow forced around fixed flow reduction elements will act to apply radial forces on the wall of the duodenum and remodel it to be large enough of a diameter to bypass the flow reduction element (see FIG. 18 for an example of such remodeling). In contrast, device body 12 of the present invention has little radial rigidity so that device body 12 or the flow around it cannot apply sufficient radial forces to remodel the duodenal tissue.

Based on video fluoroscopy gastric emptying studies, non-caloric liquids empty from the stomach faster through a relaxed small intestines (20-30 mm diameter) as compared to chyme generated by nutrient solid meals. The elements of device body 12 are sized so that they do not significantly impede the free flow of liquids through a relaxed small intestines to prevent dehydration and gastric bloating, but block chyme flow more completely when the small intestine lumen is smaller (10-20 mm diameter) as occurs during the emptying of a solid nutrient meal.

Furthermore, tether 14 allows for device body 12 to travel with the chyme in the GI tract over a set distance, and elastically stores peristaltic energy in order to impede flow of chyme through the lumen. By compartmentalizing the chyme bolus into segregated sections, device bodies 12 also inhibit the mixing of chyme in the back and forth peristaltic motions present in the small intestines after digestion of a nutrient containing meal. Such mixing is vital to mix the chyme with bile and other pancreatic juices. The lack of mixing reduces the efficiency of nutrient absorption. In contrast to prior art devices, elastic tethering of the present invention allows for relative axial motion between device body 12 and the GI tract, thereby compartmentalizing boluses of chyme between elements of device body 12 which reduces mixing effectiveness, and providing a return force to return chyme retrograde in the small intestines when tether 14 relaxes.

In a further embodiment, device 10 can comprise device body 12 in the form of a blocking element with an aperture running through it. For example, device body 12 can be a caudally pointing funnel with a 45 mm diameter open end placed in the duodenum and a 3-15 mm diameter aperture at the small end to limit the amount of chyme being delivered through device 10 to the distal parts of the small intestines. The anchoring and tethering of device 10 of this embodiment can be through any of the techniques described herein. Device 10 can have multiple such device bodies 12 along a common tether.

In yet a further embodiment, device 10 can comprise device body 12 in the form of one or more sleeves in the stomach and/or small intestine that prevent absorption of nutrients or a sleeve with a larger diameter at its proximal side relative to the diameter of the distal side to provide resistance to the flow of chyme. Such a device 10 can be fully delivered in a collapsed state through the mouth or nasopharynx using a delivery tube similar to a feeding tube. Tether 14 can be elastic or non-elastic and anchored to the esophagus, nasopharynx, nasal cavity or nares region. Using such anchoring, no endoscope is required as device body can simply be delivered to the proper position using the applicator or peristaltic motion of the small intestines.

Device 10 with a device body 12 in the form of a gastric sleeve as described above can be anchored in the stomach using in or through-tissue anchoring techniques as described herein. Device body 12 can be resident in the duodenum and connected to anchoring element 18 through one or more tethers 14, either elastic or inelastic, that runs through pyloric opening 80. In this manner, and as opposed to prior art devices that rely on the radial force of a radially expandable anchoring ring in the duodenal bulb, device body 12 of the present invention can be much less radially rigid and be collapsible to allow for lumen-occluding contractions of the duodenum that routinely occur during various phases of digestion. Excessive radial rigidity causes the duodenum to remodel around the anchoring ring of prior art devices which ends up altering the natural anatomy and leads to a procedure that is not fully reversible. The opening of the sleeve of device body 12 only has to be rigid enough to open under its own elastic force (for example a 1.5 mm diameter silicone O-ring 45 mm in diameter), but not be so rigid as to provide a normal force capable of seating an anchoring barb into the tissue. Furthermore, the proximal portion of the sleeve that makes up device body 12 can be a silicone cuff around 20-50 mm in length that has sufficient rigidity to keep the opening of the sleeve always facing the lumen and therefore resist forces trying to dislodge it and turn the opening sideways. Tether 14 (or multiple elements thereof that connect to the circumference of the sleeve opening) extend into the stomach through pyloric opening 80 and also act to keep the sleeve open and pointing in the axis of the lumen, much like the strings of a windsock keep the windsock inflated and pointing into the wind. Furthermore, the anchor approach of the present invention that utilizes an axially-oriented tether relies on the anchor efficiently resisting the axially-oriented force through tissue compression (e.g. the backstop element 16 pressing against stomach tissue), and not as is done in the prior art through radial force of a barb partially protruding into tissue and being dragged sideways by peristaltic forces, which effectively causes trauma and tissue damage on a chronic basis. In order to be effective, the latter anchoring scheme must always provide for a normal force of the barbs against the tissue. Such a force eventually causes tissue remodeling and expansion, which then further reduces the normal force in a vicious cycle that causes tissue distention to the relaxed diameter of the anchoring ring (i.e. the ring no longer provides an outward radial force) and therefore a loss of anchoring strength and a permanently distended duodenum, or perhaps even a duodenal perforation

Device body 12, whether in the form of a sleeve or any other embodiment described herein, can be sized to elastically and gently seal around the circumference of the relaxed duodenal bulb or small intestines without being able to resist the compression of the duodenal bulb or small intestines in a peristaltic wave. Alternatively, the maximum diameter of device body 12 can be less than the maximal diameter of the duodenal bulb or small intestines, therefore acting as a partial blockage or bypass from the chyme, leading to a gentler and more gradual alteration of eating behavior. Prior art sleeves (e.g. U.S. Pat. No. 5,820,584) require anchoring around the full inner circumference of the lumen and therefore act on the full flow of chyme.

In a further embodiment, device body can be a sleeve with its proximal opening under, in or above the lower esophageal sphincter for transferring all or a portion of the ingested food into the small intestines directly from the esophagus, thereby bypassing the stomach. Such a device can be anchored using an elastic or inelastic tether to the lower esophageal sphincter, or by having the tether run through the esophagus to an anchoring site in the upper esophagus, oropharynx, oral cavity, nasopharynx, nasal cavity, nasal septum or nares regions.

Device bodies of the present invention can be delivered orally and the tether fished out of the oropharynx from a trans-nasally introduced hook that then allows for anchoring of the tether in the nasal cavity, nasal septum or nares regions.

Tether 14 can be marked in such a manner so that the location of device body 12, even if not visible in the small intestines, can be inferred by relating the marks on tether 14 to known anatomical landmarks such as pyloric opening 80. Furthermore, the length of tether 14 can be fixed or adjustable after implantation using conventional or endoscopic means.

Device body 12 or portions thereof can be inflatable to vary their size. The filling port can be at the site of anchoring in the stomach or nasopharynx and a hollow tether 14 can carry fluid in and out of device body 12.

Delivery device 50 can include a measurement loop of pre-shaped wire going through the working channel of an endoscope and emerging bent 90 degrees to axis of the working channel to measure the diameter of GI lumen for the selection of a properly sized device 10 in real time.

Although use of the present device and system in stomach anchoring and modification of an eating behavior of a subject is presently preferred, device 10 of the present invention as well as delivery device 50 can be used for other purposes, including, for example, to deliver a device body which is designed for preventing or reducing reflux through the lower esophageal sphincter (LES) and thus can be used for treatment of gastroesophageal reflux disease (GERD).

Such a device body can be shaped as a flat disc, a cone, or any other configuration as described for device body 12 which can be loaded into a suitable delivery device 50 modified for use in GERD treatment. For example, the lower end of the tether of a GERD prevention device 10 can be attached to stomach tissue just below the LES using delivery device 50 and the upper end of tether 14 can be attached higher up in the esophagus, oropharynx, nasopharynx, oral cavity, nasal cavity or nasal septum. The upper anchoring prevents device body 12 from migrating downward due to peristalsis. The lower anchoring prevents device body 12 from migrating back up through the esophagus due to belching or vomiting. Device body 12, in the form of one or more cones or cylinders, can be attached to tether 14 just above the lower anchoring point so that device body 12 traverses the LES. Example 7 below further describes anchoring of a GERD device in the GI tract of a pig.

Although specific embodiments of the present invention were presented individually or assembled into specific systems, all individual elements of the device or associated positioning and delivery systems can be present multiple times and in various configurations in a modular manner in all of embodiments of the present invention.

As used herein the term “about” refers to ±10%.

Additional objects, advantages, and novel features of the present invention will become apparent to one ordinarily skilled in the art upon examination of the following examples, which are not intended to be limiting. Additionally, various embodiments and aspects of the present invention as delineated hereinabove and as claimed in the claims section below find experimental support in the following examples.

Reference is now made to the following examples, which together with the above descriptions, illustrate the invention in a non limiting fashion.

### Example 1

### An Antrum-Anchored Gastric Device in Pigs Using Non-Elastic Anchoring

System 100 as illustrated in FIGS. 5a-c was used to implant device 10 into the antrums of three live pigs weighing 40 kilos. The pigs were anesthetized and an Olympus GIF130 gastroscope with attached delivery device 50 was utilized to deliver and anchor device 10 as illustrated in FIG. 4b to the distal antrum of the pigs. Delivery device 50 includes vacuum chamber 52 for grabbing wall tissue via vacuum applied from an attached vacuum hose (which runs outside and along the endoscope) and a device chamber 56 for holding the hollow device body 12. Delivery device 50 is configured such that a tissue penetrating element (delivery needle 206) is delivered through a working channel in the gastroscope and can penetrate through a tissue fold held in vacuum chamber 52 and into device body 12 which in this case is a silicone cylinder with rounded ends and an enclosed hollow portion. Delivery device 50 is designed so as to not fully block the light and camera lens fitted on the gastroscope and thus it provides a user with a clear view of the procedure, including the tissue invaginating into vacuum chamber 52.

Device body 12 is secured via frictional forces within device chamber 56 at the front portion of the delivery device. A force of 2 Newtons is sufficient to slide device body 12 out of device chamber 56 in a direction normal to the endoscope axis. Anchoring element 18 (in this case a t-bar anchor) attached to tether 14 is secured within a groove formed in delivery needle 206. Delivery needle 206 is disposed within the working channel of the gastroscope while the backstop element 16 is secured within a side pocket in delivery device 50. A force of 0.5 Newtons is sufficient to slide backstop element 16 out of pocket 60.

Device 10 was anchored to antral wall region of stomachs of female pigs using tether 14 made of 2-0 monofilament polypropylene suture material fitted with an 8 mm diameter by 1 mm thick silicone backstop element 16 on one end and a 6 mm long 21G diameter metal t-bar anchoring element 18 on the other end of tether 14.

System 100 was guided through the mouth of an anesthetized pig through the esophagus and into the stomach and positioned close to the pylorus in the antrum. Vacuum was used to pull a section of the wall tissue into vacuum chamber 52. Once a wall tissue fold fully filled vacuum chamber 52, delivery needle 206 was pushed through the wall tissue fold under direct visual guidance. Anchoring element 18 and attached tether 14 were advanced through the wall tissue and into device body 12. An internal plunger (not shown) was pushed thereby deploying the t-bar anchoring element 18 out of delivery needle 206 and into the hollow space within device body 12. Delivery needle 206 was retracted leaving the t-bar anchoring element 18 secured against an internal seating surface inside device body 12. Vacuum was released and the gastroscope was moved away from the tissue releasing device body 12 (as is demonstrated in FIG. 5c) from device chamber 56 and backstop element 16 from pocket 60.

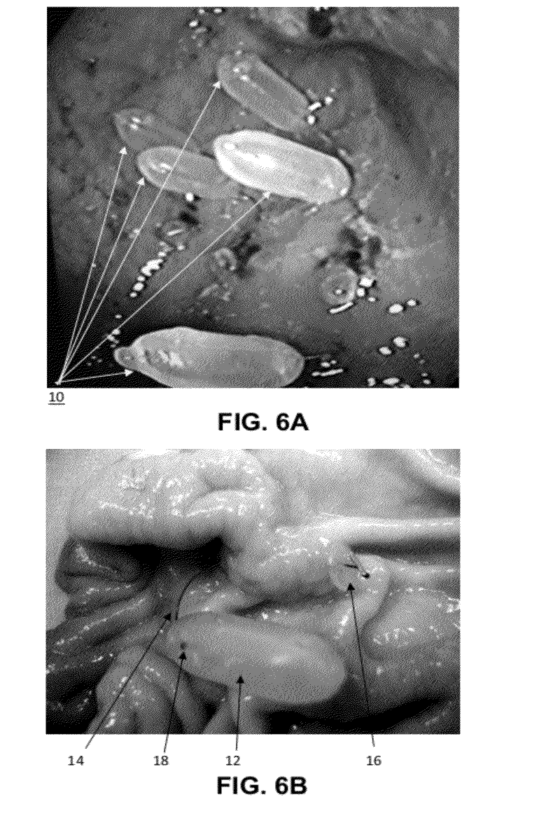

The above described procedure was repeated several times for each pig. FIG. 6a shows an endoscopic view of five such devices anchored in the stomach of a single pig that resided with no complications in the stomach for at least 8 weeks.

One of the pictured devices was removed endoscopically using a standard suture cutter introduced in the working channel of the standard endoscope. Tether 14 was cut close to the entry point of tether 14 into the device body and then detached device body 12, tether 14 and backstop element 16 were separately removed from the mouth of the pig with endoscopically introduced standard forceps and basket accessories. Device 10 was this removed completely with endoscopic means with no foreign bodies, sutures or anchor elements left in the animal and the tract formed by the suture healed within a day. All parts of device 10 are sized that should they detach on their own, they can all safely pass through the pylorus (2.5 cm diameter object or less pass through it) and will be evacuated harmlessly in the feces.

One of the animals carrying a device was sacrificed at the end of an 8 week period and tissue around (and including) the anchoring site was harvested (FIG. 6b), and analyzed. No appreciable tissue changes were observed in the mucosa and tunica muscularis indicating that long term anchoring using the above described procedure produces no visible erosion, tissue reaction or other adverse affects.

### Example 2

### An Antrum-Anchored Gastric Device in Human Patients

System 100 as illustrated in FIGS. 5a-c was used to implant device 10 into the antrums of 2 human patients. Patient 1 weighed 120 kilos (BMI 35.1) and patient 2 weighed 137 kilos (BMI 35.7) before the procedure. The patients were sedated and an Olympus GIF130 gastroscope with attached delivery device 50 was utilized to deliver and anchor device 10 as illustrated in FIG. 4b to the distal antrum of the patients. The rest of the procedure was performed as per Example 1 above. In patient 1, the anchoring site of device 10 was approximately 9 cm away from the pylorus and in patient 2 around 5 cm away from the pylorus.

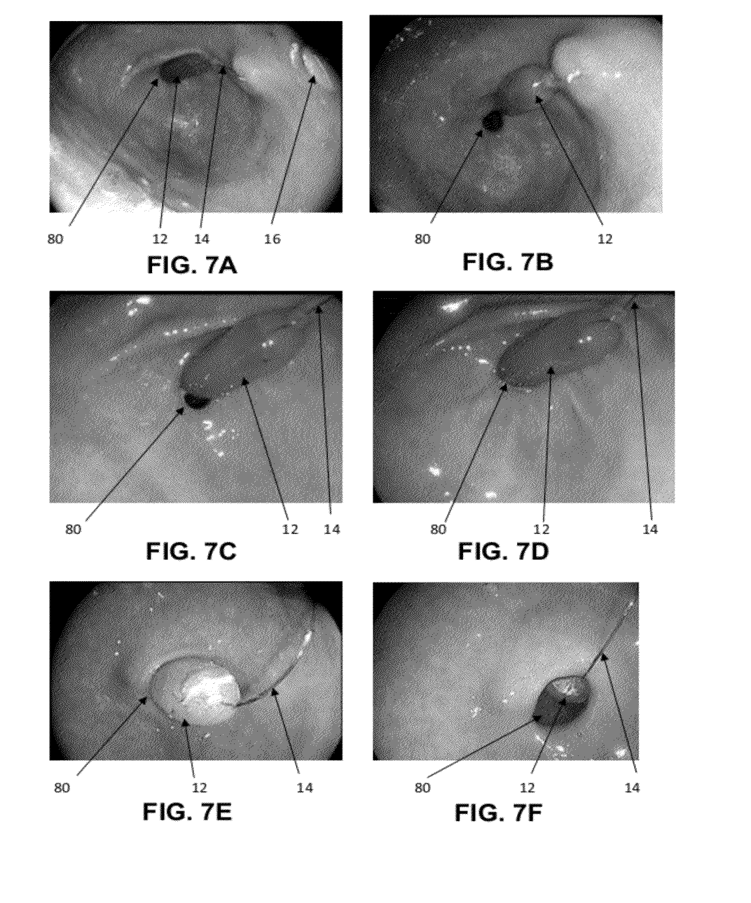

FIG. 7a shows device 10 anchored in the distal antrum of one of the patients with backstop element 16, tether 14, device body 12 and pyloric opening 80 all visible. FIG. 7b shows device body 12 approaching pyloric opening 80. FIG. 7c shows device body 12 partially blocking pyloric opening 80. FIG. 7d shows device body 12 full blocking pyloric opening 80. FIG. 7e shows device body 12 partially passing (and fully blocking in the process) pyloric opening 80 with tether 14 about to restrain the forward motion of device body 12. FIG. 7e clearly illustrates how the largest portion of device 10, namely device body 12, is configured to fully pass through the pyloric opening and continue into the duodenum where it not restrained from doing so by tether 14 and backstop element 16. FIG. 7f shows device body 12 inside the duodenal bulb just behind pyloric opening 80 and restrained from further antegrade motion by a taught tether 14. During the course of digestion and peristalsis, device body 12 would routinely shuttle back and forth from positions 7a, 7b, 7c, 7d, 7e, and 7f thereby providing intermittent contact with the stomach/duodenal tissue and partial and intermittent blockage of the pyloric opening, causing delayed gastric emptying and early satiety.

Both patients felt no pain within a day after the procedure and reported early satiety and feeling of staying full longer while eating smaller portions of food and eating it slower. Patient 1 kept device 10 implanted for 9 weeks, at which point it was endoscopically removed. Patient 1 lost 6 kilos in this time frame for a final BMI of 33.3. Patient 2 found an intact device 10 in the toilet after 10 weeks but had lost 13 kilos in this time frame for a final BMI of 32.3.

A follow up endoscopy showed that both patients had no lasting signs or damage to the stomach tissue and that the implantation of device 10 using delivery device 50 is both easy and safe.

From the loss of device 10 in patient 2, the inventor learned that tether 14 made of polypropylene had eroded through the stomach tissue due to ongoing peristalsis and contraction waves in the distal antrum. A second embodiment of device 10, as illustrated in FIG. 8, has tether 14 which is made of silicone as is much softer and more elastic that polypropylene and hence is not as likely to erode the stomach tissue and therefore is designed to be implanted longer than device 10 as described in the current example. Implantation of device 10 as illustrated in FIG. 8 is described in the next example.

### Example 3

### An Antrum-Anchored Gastric Device in Pigs Using Elastic Anchoring

System 100 as illustrated in FIGS. 9a-b and 10a-d was used to implant device 10 into the antrums of three live pigs weighing 40 kilos. The pigs were anesthetized and an Olympus GIF130 gastroscope with attached delivery device 50 was utilized to deliver and anchor device 10 as illustrated in FIG. 8a-b to the distal antrum of the pigs. Device 10 is made completely out of shore A 60 silicone with a hollow 8 mm diameter front ball with 0.5 mm wall thickness, a 1.5 mm diameter stem between the disks, a 1.2 mm diameter tether and a 0.9 mm diameter silicone t-anchor overmolded over an 8 mm long stainless steel wire 0.25 mm in diameter with 1 mm long 0.9 mm diameter stainless steel spacers at the ends to keep the wire centered in the mold during the casting of the silicone.

Delivery device 50 includes vacuum chamber 52 for grabbing wall tissue via vacuum applied from within the working channel of endoscope 68 and pocket 60 to hold disk 74 which is part of device body 12. Delivery device 50 is configured such that a tissue piercing element (delivery needle 206) is delivered through an external needle guide 58 which runs parallel to endoscope 68 and can penetrate through a tissue fold held in vacuum chamber 52 and through washer 208. Delivery device 50 is designed so as to not block the light and camera lens fitted on the gastroscope and thus it provides a user with a clear view (greater than 75% of the full field of view) of the procedure, including distance marker 83 superimposed on pyloric opening 80.

Device body 12 is secured via frictional forces within pockets 60 and a normal force of 2 Newtons is sufficient to slide device 10 out of delivery device 50. Anchoring element 18 (in this case a t-bar anchor) attached to tether 14 is secured within a groove formed in delivery needle 206.

System 100 was guided through the mouth of an anesthetized pig through the esophagus and into the stomach and positioned close to the pylorus in the antrum. Vacuum was used to pull a section of the wall tissue into vacuum chamber 52. Once a wall tissue fold fully filled vacuum chamber 52, delivery needle 206 was pushed through the wall tissue fold under direct visual guidance. Anchoring element 18 and attached tether 14 were advanced through the wall tissue, through an 8 mm diameter and 0.5 mm thick silicone washer 208, and into anchor release chamber 84. An internal plunger (not shown) was pushed thereby deploying the t-bar anchoring element 18 out of delivery needle 206 and into the hollow space within anchor release chamber 84. Delivery needle 206 was retracted leaving the t-bar anchoring element 18 secured against washer 208. Vacuum was released and the gastroscope was moved away from the tissue releasing device body 12 (as is demonstrated in FIG. 10d) from pocket 60 and anchoring element 18 from anchor release chamber 84 though slot 85.

The above described procedure was repeated several times for each pig. FIG. 11a shows an endoscopic view of device 10 anchored in the stomach of a single pig close to pyloric opening 80. FIG. 11b shows an endoscopic view of device 10 that passes partially through pyloric opening 80 and disk 74 partially blocking pyloric opening 80 with stem 76 connecting two disk 74 elements. If the proximal disk 74 is passed through to the duodenum, then a more distal disk 74 is automatically advanced to partially block pyloric opening 80, making device 10 more effective at blocking the pylorus irrespective of the exact position of anchoring of device 10, the length of tether 14 or the orientation of device 10 in the stomach. FIGS. 11c and 11d show device 10 after 2 weeks in the stomach of a pig and show the lack of erosion on the mucosal surface and through the tissue tract that tether 14 passes through indicating that device 10 is not likely to erode out of stomach tissue nor is it likely to damage the stomach tissue.

One of the pictured devices was removed endoscopically using a standard suture cutter introduced in the working channel of the standard endoscope. Tether 14 was cut close to the entry point of tether 14 into the device body and then detached device body 12, tether 14 and backstop element 16 were separately removed from the mouth of the pig with endoscopically introduced standard forceps and basket accessories. Device 10 was this removed completely with endoscopic means with no foreign bodies, sutures or anchor elements left in the animal and the tract formed by the suture healed within a day. All parts of device 10 are sized that should they detach on its own, they can all safely pass through the pylorus (2.5 cm diameter object or less pass through it) and will be evacuated harmlessly in the feces.

### Example 4

### Implantation of a Pyloric-Anchored Gastric Device in Live Pigs

Delivery device 50 illustrated in FIG. 13a-b was used to anchor an eating behavior modification device of the present invention to the pyloric sphincter muscle of an anesthetized 40 kilo female pig. Delivery device 50 was positioned to straddle the ridge of the tissue surrounding pyloric opening 80 as is illustrated in FIG. 13b. Delivery needle 206 was pushed through needle guide 58 to the distal side of the pyloric muscle where anchoring element 18 was deployed, keeping device body 12 in front of pyloric opening 80 to partially block the flow of chyme from the stomach to the duodenum. No vacuum was required in this procedure which was done solely under visual guidance. The pig was sacrificed after two weeks and the tissue showed no sign of erosion or inflammation.

### Example 5

### An Antrum-Anchored Gastric Device in Pigs Resident in the Duodenum

System 100 as illustrated in FIGS. 3a-c was used to implant device 10 into the antrums of three live pigs weighing 40 kilos. The pigs were anesthetized and an Olympus GIF130 gastroscope with attached delivery device 50 was utilized to deliver and anchor device 10 as illustrated in FIG. 3a-c to the distal antrum of the pigs. Device 10 is made completely out of shore A 60 silicone with a solid 10 mm diameter 25 mm long pill shaped solid device body 12 connected through tether 14 which is made of 1.5 mm diameter silicone and connected to a 20 mm long polyester non-absorbable 2-0 suture forming a second non-elastic portion of tether 14 which in turn was connected to anchoring element 18 which was made of a 1 mm diameter×6 mm long stainless steel tube resting against a silicone washer 208.

Delivery device 50 and the implantation procedure was performed as per Example 1 above except that device body 12 was freely dragged behind endoscope 68 and not held in pocket 60. Anchoring element 18 inside delivery needle 206 was the only means of attaching device 10 to delivery device 50. Device 10 was anchored 3-5 cm from pyloric opening 80. The pigs recovered within half of a day and starting eating lower quantities of food than before the procedure.

One of the pigs was sacrificed just 1 week after implantation and a histological analysis showed device body 12 resting in the proximal duodenum as illustrated in FIG. 18. The proximal duodenal tissue around device body 12, shown as region 92, had mucosal ridges that were flattened by device body 12. This region had started to remodel itself around device body 12. Duodenal bulb 90 which was upstream and regions of the duodenum downstream of where device body 12 was located did not show this mucosal flattening or remodeling. Additional experiments with device bodies that were more easily collapsible (e.g. a radial force of 1 Newton in any direction could cause the device to completely collapse) and tethered with fully elastic tethers as described in Example 6 below did not cause the duodenal tissue, which is thinner and more sensitive than the stomach tissue, to flatten out, erode, or remodel. Therefore, Example 5 illustrates the importance of a crushable and easily collapsible device body 12 and elastic tethering that allows for relative motion between device body 12 and the duodenum if device body 12 is intended to be resident in the duodenum over the long term.

### Example 6

### A Device Anchored in the Antrum of Pigs

System 100 as illustrated in FIG. 19a was used to implant device 10 as illustrated in FIGS. 16b-c into the antrums of eight live pigs weighing 40 kilos each. The pigs were anesthetized and an Olympus GIF130 gastroscope with attached delivery device 50 was utilized to deliver and anchor device 10 to the distal antrum of the pigs. Device 10 is made completely out of shore A 50 silicone with two crushable 20 mm diameter parabolic umbrella-shaped device bodies 12 connected through tether 14 which is made of 1.2 mm diameter silicone tether 14 which in turn was connected to anchoring element 18 which was made of a 0.33 mm diameter×8 mm long stainless steel pin against silicone washer 208.

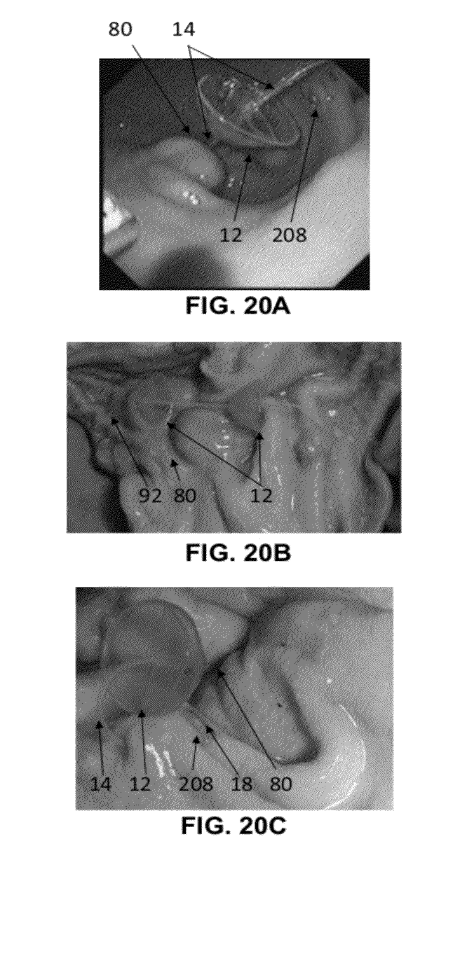

Delivery device 50 and the implantation procedure was performed as per Example 1 above except that device body 12 was dragged alongside endoscope 68 and not held in pocket 60. Anchoring element 18 inside delivery needle 206 and front tether 70 which was friction fit inside restraining strap 96 were used to attach device 10 to delivery device 50. Device 10 was anchored 3-5 cm from pyloric opening 80 and then detached from endoscope 68 by withdrawal of endoscope 68 and the resulting detachment of front tether 70 from restraining strap 96. The pigs recovered within half of a day. FIG. 20a shows an endoscopic view of the proximal device body 12 in the antrum partially blocking pyloric opening 80. Tether 14 runs through pyloric opening 80 into the duodenum where the distal device body 12 is resident (not visible), thereby showing the stable anchoring and positioning of device 10 as intended.

Two of the pigs were sacrificed 3 months after implantation and a histological analysis showed one device body 12 resident in the proximal duodenum and one device body resident in the distal antrum, as illustrated in FIG. 20 b-c. FIG. 20c illustrates the lack of inflammation and the stability of the elastic tether anchoring scheme of the present invention. FIG. 20b illustrates an excised pig stomach and duodenal region split and spread open with the proximal duodenal tissue around device body 12, shown as region 92, having mucosal ridges that were undisturbed by device body 12 throughout the 3 month experiment, in contrast to example 5 above. Therefore, Example 6 further illustrates the importance of a crushable and easily collapsible device body 12 and elastic tethering that allows for relative motion between device body 12 and the duodenum/antrum if device 10 is intended to reside in the duodenum/antrum over the long term.

### Example 7

### Implantation of an Acid Shield Device in the Lower Esophageal Sphincter of a Live Pig

The present invention can also be used to treat gastric esophageal reflux disease (GERD). Device body 12 is conical or flat elastic disk made of silicone that is mounted at the forward end of delivery device 50 as pictured in FIG. 13a-b. Tether 14 is 25 mm long and 1.5 mm diameter silicone that is over-molded on top of a metal t-bar at one end to form anchoring element 18 which is mounted inside a slot in delivery needle 206. Backstop element 16 is a 6 mm diameter 1 mm thick silicone disk at the rear end of tether 14 that ends up resting as a backstop on the esophageal tissue surface at the site of the entry hole of delivery needle 206. Delivery device was mounted on a single channel Olympus GIF130 gastroscope and guided through the mouth of an anesthetized 40 kg pig through the esophagus close to the entrance to the stomach. The lower esophageal sphincter (LES) was allowed to invaginate into the L-shaped applicator through natural muscle contraction, thereby placing a tissue fold between the needle and device body 12 which is mounted at the distal end of delivery device 50. Alternatively, a vacuum chamber can be used to pull in the wall of the tissue as per Examples 1-5, but in this case the natural invagination of the LES helped to confirm the correct position of implantation. At higher and lower points relative to the LES the esophagus or stomach tissue will not naturally invaginate into this space.

Delivery needle 206 was pushed through the tissue fold and through the center of device body 12 into an anchor release chamber 84 at the distal tip of delivery device 50 that was in the stomach. Anchoring element 18, which ran through device body 12, was deployed using an internal pushrod. Delivery needle 206 was retracted and endoscope 68 was withdrawn from the mouth of the pig leaving the tether elastically anchoring the device body to the bottom surface of the LES. In such a configuration, the device body acts as a one-way “flap valve” and prevents acid from entering the esophagus thereby reducing or eliminating GERD while not preventing or hindering the entrance of food into the stomach.

Following removal of the device, no appreciable tissue changes were observed in the mucosa and tunica muscularis indicating that long term anchoring using the above described procedure produces minimal tissue reaction and no adverse affects.

An alternative anchoring scheme in which backstop 16 is positioned higher in the esophagus, oral cavity, nasopharynx, or nasal cavity could also be implemented by simply increasing the length of tether 14 and anchoring backstop element 16 in a separate procedure to the desired tissue. The lower anchoring site in the area of anchor 18 would prevent device body 12 from being refluxed into the esophagus and out the mouth in the event of belching or vomiting. The upper backstop element 16, by forces applied via tether 14, would prevent device body 12 from migrating into the stomach due to peristaltic forces, thereby keeping it properly positioned in the region of the LES.

A further alternative would be to apply anchor 18 to stomach tissue under the LES using delivery device 50 pictured in FIG. 19 and to position device body 12 on tether 14 between anchor 18 and backstop element 19 so that device body 12 is stably positioned in the region of the LES without the need to perform a needle puncture in the esophagus or LES.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A device for treating a gastrointestinal disorder of a subject comprising a tether having an end portion adapted for anchoring to a tissue of a GI tract and at least one device body attached along a length of said tether, said at least one device body being a normally-open, elastically-compressible structure capable of:
(i) at least partially occluding a lumen of said GI tract in said normally-open state; and
(ii) passing through said GI tract following detachment of said tether from said tissue of said GI tract.

2. The device of claim 1, wherein said at least one device body is configured for at least partially blocking an antral, pyloric and/or duodenal region of said GI tract and further wherein said end portion is anchorable to a tissue of a stomach and a length of said tether is selected for positioning said at least one device body 7 cm or less from said pylorus.

3. The device of claim 1, wherein said device body is shaped as a hollow sleeve, open cone or umbrella.

4. The device of claim 3, wherein said hollow sleeve, open cone or umbrella comprises a web to resist inversion.

5. The device of claim 1, wherein said device body is a disk having a wall thickness of 1 mm or less.

6. The device of claim 1, wherein said at least one device body has a maximum displaced volume of 1 cubic centimeter.

7. The device of claim 2, wherein at least a portion of said tether can reversibly increase in length by least 25%.

8. The device of claim 2, wherein said at least one device body is capable of moving between a stomach and a duodenum when said tether is anchored to said tissue of said stomach.

9. The device of claim 1, wherein said at least one device body has a maximal cross sectional area of 10 square centimeters.

10. The device of claim 1, wherein said at least one device body is configured to collapse to its minimal contained volume under a force of less than 1 Newton.

11. The device of claim 1, wherein said at least one device body can elastically deform from a contained volume of zero to a contained volume of up to 30 cm³.

12. The device of claim 1, wherein all portions of the device pass through the GI tract following detachment of the device from said tissue of said GI tract.

13. A system for treating a gastrointestinal disorder comprising:
(a) a device including at least one device body attached along a length of a tether having an end portion anchorable to a tissue; and
(b) a delivery apparatus including:
(i) a tissue penetrating element for delivering said end portion of said tether through a tissue of a gastrointestinal wall; and
(ii) a distance marker for determining a tissue site for delivering said end portion of said tether through said tissue such that said end portion of said tether is positioned at a predetermined distance from a tissue landmark.

14. The system of claim 13, wherein said delivery apparatus includes a vacuum chamber for suctioning said tissue in a direction normal to an axis of movement of said tissue penetrating element.

15. The system of claim 13 wherein said tissue penetrating element includes a pushrod positioned within a lumen thereof and further wherein said delivery apparatus includes a handle configured for sequentially displacing said tissue penetrating element and said pushrod in one operator motion.

16. A method of modifying an eating behavior of a subject comprising:
(a) delivering the device of claim 1 to the stomach of the subject; and
(b) delivering said end portion of said tether through tissue of said stomach such that said end portion of said tether is positioned at a predetermined distance from a tissue landmark thereby modifying an eating behavior of the subject.

17. The method of claim 16, wherein (b) is effected such that an opening of said device body is oriented facing the oral direction during gastric emptying.

18. The method of claim 16, wherein (a) is effected by sucking tissue into a vacuum chamber in a direction normal to the axis of movement of a tissue penetrating element.

19. The method of claim 16, wherein said delivering said end portion of said tether through tissue of said stomach is effected using a distance marker for determining a tissue site for penetration relative to a tissue landmark.

20. The method of claim 19, wherein said tissue landmark is a pylorus.
